(19) United States
(12) Patent Application Publication
(10) Pub. No.: US 2018/0318427 A1
(43) Pub. Date: Nov. 8, 2018

(54) PHARMACEUTICAL FORMULATIONS COMPRISING INSULIN OR INSULIN ANALOGS CONJUGATED TO FUCOSE

(71) Applicant: MERCK SHARP & DOHME CORP., RAHWAY, NJ (US)

(72) Inventors: Shuai Shi, Whippany, NJ (US); Valentyn Antochshuk, Cranford, NJ (US); Joseph Rizzo, Howell, NJ (US)

(73) Assignee: MERCK SHARP & DOHME CORP., RAHWAY, NJ (US)

(21) Appl. No.: 15/966,602

(22) Filed: Apr. 30, 2018

Related U.S. Application Data
(60) Provisional application No. 62/502,020, filed on May 5, 2017.

(51) Int. Cl. A61K 47/54; A61K 47/02; A61K 47/10; A61K 47/12; A61K 38/28; A61P 3/10

(52) U.S. Cl. CPC A61K 47/549; A61K 47/02; A61P 3/10; A61K 47/12; A61K 38/28; A61K 47/10

(57) ABSTRACT

Disclosed is a pharmaceutical formulation comprising an insulin or insulin analog molecule covalently attached to at least one branched linker having a first arm and second arm, wherein the first arm is linked to a first ligand that includes a first saccharide and the second arm is linked to a second ligand that includes a second saccharide and wherein the first saccharide is fucose suitable for parenteral or subcutaneous administration and which is suitable for use in controlling post-prandial glucose levels in an individual.

### CROSS-REFERENCE TO RELATED APPLICATIONS

This application claims priority to U.S. Provisional Patent Application No. 62/502,020, filed May 5, 2017.

### FIELD OF THE INVENTION

The present invention relates to a pharmaceutical formulation comprising an insulin or insulin analog molecule covalently attached to at least one branched linker having a first arm and second arm, wherein the first arm is linked to a first ligand that includes a first saccharide and the second arm is linked to a second ligand that includes a second saccharide and wherein the first saccharide is fucose and which is suitable for use in controlling post-prandial glucose levels in an individual.

### BACKGROUND OF THE INVENTION

Nearly a century has passed since the discovery of insulin and its rapid introduction into clinical use. However, the remarkable historical success of exogenous insulin therapy has been tempered by the risk for hypoglycemia. This risk of insulin-induced hypoglycemia stands as one of the major barriers to the achievement of tight glycemic control (P. E. Cryer, “Hypoglycaemia: The limiting factor in the glycaemic management of Type I and Type II Diabetes”, 45 Diabetologia 937-948 (2002)). To mitigate risk for hypoglycemia, a focus of innovation has been to improve insulin pharmacokinetics (PK) (Geremia B. Bolli & J. Hans DeVries, “New Long-Acting Insulin Analogs: From Clamp Studies to Clinical Practice”, 38 Diabetes Care 541-543 (2015); Lutz Heinemann & Doublas B. Muchmore, “Ultrafast-Acting Insulins: State of the Art”, 6(4) J. Diabetes Sci. & Tech. 728-742 (July 2012); Alexander N. Zaykov et al., “Pursuit of a perfect insulin”, 15 Nature Reviews 425-439 (June 2016); M. Brownlee & A. Cerami, “A glucose-controlled insulin-delivery system: semisynthetic insulin bound to lectin”, 206 Science 1190-1191 (Dec. 7, 1979)). Long-acting “basal” insulin innovations have sought to sustain insulin absorption and reduce variability of plasma PK to provide constancy in control of hepatic glucose production (HGP). Prandial insulin innovations, meanwhile, have been just the opposite: to achieve a more rapid peak of action (Lutz Heinemann & Doublas B. Muchmore, “Ultrafast-Acting Insulins: State of the Art”, 6(4) J. Diabetes Sci. & Tech. 728-742 (July 2012)). However, efforts to improve insulin PK do not change the intrinsically narrow therapeutic index of native insulin nor enable exogenous insulin to autonomously modulate action in the face of descending plasma glucose and mitigate risk for hypoglycemia. A persistent risk for hypoglycemia can cause patients to be cautious with insulin dosing, in essence to modestly under-dose, aggravating risk for the development of micro- and macrovascular complications. A key impetus to create closed-loop insulin delivery is to establish real-time communication about ambient glucose that can inform and modulate exogenous insulin delivery.

Another approach to creating communication between exogenously administered insulin and a patient's blood glucose is to engineer insulin so that it will intrinsically respond to fluctuations in ambient glucose. The notion of glucose responsive insulin (GRI) was proposed nearly 40 years ago (M. Brownlee & A. Cerami, “A glucose-controlled insulin-delivery system: semisynthetic insulin bound to lectin”, 206 Science 1190-1191 (Dec. 7, 1979)). A number of attempts at creating a GRI have been reported and most of these have sought to exploit the concept of incorporating insulin into a matrix containing glucose sensitive chemical “triggers” that affect release of insulin from a subcutaneous depot (Jicheng Yu et al., “Microneedle-array patches loaded with hypoxia-sensitive vesicles provide fast glucose-responsive insulin delivery”, 112(27) PNAS (Jul. 7, 2015); Danny Hung-Chieh Chou et al., “Glucose-responsive insulin activity by covalent modification with aliphatic phenylboronic acid conjugates”, 112(8) PNAS 2401-2406 (Feb. 24, 2015); Junling Guo et al., “Boronate-Phenolic Network Capsules with Dual Response to Acidic pH and cis-Diols”, 4 Adv. Healthcare Mater. 1796-1801 (2015); Xiuli Hu et al., “H₂O₂-Responsive Vesicles Integrated with Transcutaneous Patches for Glucose-Mediated Insulin Delivery”, 11(1) ACS Nano. 613-620 (Jan. 24, 2017)). However, most of the aforementioned approaches have met with limited success primarily because of the challenges associated with attaining glucose modulation of insulin action across a relatively small range of ambient glucose concentrations. An alternative strategy for exploring glucose responsiveness by exploiting lectin based clearance was reported by Zion and Lancaster and disclosed in PCT International Patent Application Publication No. WO2010/088294 and U.S. Pat. No. 9,579,391.

Lectins recognize and bind carbohydrate domains of glycoproteins. All mammalian species possess circulating and cell-based lectins that function in immune surveillance as well as clearance of glycoproteins (Maureen E. Taylor & Kurt Drickamer, “Convergent and divergent mechanisms of sugar recognition across kingdoms”, 28 Current Opinion in Structural Biology 14-22 (2014); Kurt Drickamer & Maureen E. Taylor, “Recent insight into structures and functions of C-type lectins in the immune system”, 34 Current Opinion in Structural Biology 26-34 (2015)). The mannose receptor family is a subgroup of the C-type lectin superfamily that contains multiple C-type lectin domains (CTDL) within a single protein backbone. Among these, mannose receptor C Type 1 (MR; MR; CD206, MMR) is the prototypical member of the mannose receptor family of transmembrane lectins. A main function of MR is to recognize endogenous senescent and “waste” proteins tagged for destruction as well as pathogens identified by their surface glycan, and through preferential binding to terminal mannose (e.g. paucimannose such as Man3), followed by L-fucose, and N-acetylglucosamine, to bind these ligands and deliver these for lysosomal degradation (Ann M. Kerrigan & Gordon D. Brown, “C-type lectins and phagocytosis”, 214 Immunobiology 562-575 (2009); Reiko T. Lee et al., “Survey of immune-related, mannose/fucose-binding C-type lectin receptors reveals widely divergent sugar-binding specificities”, 21(4) Glycobiology 512-520 (2011); Luisa Martinez-Pomares, “The mannose receptor”, 92 J. Leukocyte Biology 1177-1186 (December 2012); Sena J. Lee et al., “Mannose Receptor-Mediated Regulation of Serum Glycoprotein Homeostasis”, 295 Science 1898-1901 (Mar. 8, 2002)). MR is an abundant lectin, residing at a high level in hepatic sinusoidal endothelial cells (HSEC), and on certain macrophages and dendritic cells. It is well conserved homology across species (Kurt Drickamer & Maureen E. Taylor, “Recent insight into structures and functions of C-type lectins in the immune system”, 34 Current Opinion in Structural Biology 26-34 (2015)). MR functions as a high-capacity system for clearance of substrates containing mannose and/or N-acetylglucosamine motifs without eliciting an immune response or cytokine release. Glucose has a low affinity for MR but can compete with other MR ligands. Chimeric insulin analogs have been described that when plasma glucose is within the euglycemic or hypoglycemic range, a substantial fraction of the insulin analog is cleared by MR, lessening availability for interaction with the insulin receptor (IR) whereas with progressively higher ambient glucose, a reduced fraction of the insulin analog will be cleared by MR, creating a higher circulating concentration and greater interaction with the IR.

### SUMMARY OF THE INVENTION

The present invention provides pharmaceutical formulations comprising an insulin oligosaccharide conjugate that is suitable for parenteral or subcutaneous administration and that is suitable for use in controlling post-prandial glucose levels in an individual.

The present invention provides a pharmaceutical formulation comprising or consisting essentially of an insulin oligosaccharide conjugate, sodium phosphate buffer, zinc salt, halide, glycerin, and phenolic compound, wherein the formulation has a pH in the range of 6.2 to 7.0; wherein the insulin oligosaccharide conjugate comprises an insulin or insulin analog molecule covalently attached to at least one branched linker having a first arm and second arm, wherein the first arm is linked to a first ligand that includes a first saccharide and the second arm is linked to a second ligand that includes a second saccharide; and, wherein the first saccharide is fucose.

In particular embodiments, the pharmaceutical formulation comprises about 4 mg/mL to 40 mg/mL insulin oligosaccharide conjugate, a molar equivalent zinc salt to the insulin oligosaccharide conjugate monomer ratio of about 0.35 to 1.0, about 5 to 50 mM sodium phosphate buffer, about 5 to 100 mM halide, about 16.0 mg/mL glycerin, and about 2.8 mg/mL phenolic compound.

In particular embodiments, the pharmaceutical formulation comprises about 4 mg/mL insulin oligosaccharide conjugate, a molar equivalent zinc salt to the insulin oligosaccharide conjugate monomer ratio of about 0.45, about 7 mM sodium phosphate buffer, about 15 mM halide, about 16.0 mg/mL glycerin, and about 2.8 mg/mL phenolic compound.

In particular embodiments, the zinc salt is selected from zinc acetate and zinc chloride (ZnCl₂). In a particular embodiment, the zinc salt is zinc chloride.

In particular embodiments, the halide is sodium chloride.

In particular embodiments, the phenolic compound is m-cresol.

In particular embodiments, the insulin oligosaccharide conjugate comprises general formula (I):

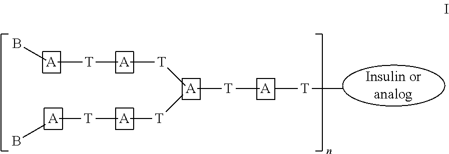

wherein:

each occurrence of

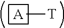

represents a potential repeat within a branch of the conjugate;

each occurrence of [symbol] is independently a covalent bond, a carbon atom, a heteroatom, or an optionally substituted group selected from the group consisting of acyl, aliphatic, heteroaliphatic, aryl, heteroaryl, and heterocyclic;

each occurrence of T is independently a covalent bond or a bivalent, straight or branched, saturated or unsaturated, optionally substituted C₁₋₃₀ hydrocarbon chain wherein one or more methylene units of T are optionally and independently replaced by —O—, —S—, —N(R)—, —C(O)—, —C(O)O—, —OC(O)—, —N(R)C(O)—, —C(O)N(R)—, —S(O)—, —S(O)₂—, —N(R)SO₂—, —SO₂N(R)—, a heterocyclic group, an aryl group, or a heteroaryl group;

each occurrence of R is independently hydrogen, a suitable protecting group, or an acyl moiety, arylalkyl moiety, aliphatic moiety, aryl moiety, heteroaryl moiety, or heteroaliphatic moiety;

—B is -T-L^{B}-X;

each occurrence of X is independently a ligand;

each occurrence of L^{B} is independently a covalent bond or a group derived from the covalent conjugation of a T with an X; and,

wherein n is 1, 2, or 3, with the proviso that the insulin is conjugated to at least one linker in which one of the ligands is fucose.

In particular embodiments, the insulin oligosaccharide conjugate comprises general formula (II):

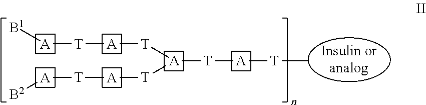

wherein:

each occurrence of

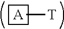

represents a potential repeat within a branch of the conjugate;

each occurrence of [symbol] is independently a covalent bond, a carbon atom, a heteroatom, or an optionally substituted group selected from the group consisting of acyl, aliphatic, heteroaliphatic, aryl, heteroaryl, and heterocyclic;

each occurrence of T is independently a covalent bond or a bivalent, straight or branched, saturated or unsaturated, optionally substituted C₁₋₃₀ hydrocarbon chain wherein one or more methylene units of T are optionally and independently replaced by —O—, —S—, —N(R)—, —C(O)—, —C(O)O—, —OC(O)—, —N(R)C(O)—, —C(O)N(R)—, —S(O)—, —S(O)₂—, —N(R)SO₂—, —SO₂N(R)—, a heterocyclic group, an aryl group, or a heteroaryl group;

each occurrence of R is independently hydrogen, a suitable protecting group, or an acyl moiety, arylalkyl moiety, aliphatic moiety, aryl moiety, heteroaryl moiety, or heteroaliphatic moiety;

—B1 is -T-L^{B1}-fucose,

wherein L^{B1} is a covalent bond or a group derived from the covalent conjugation of a T with an X;

—B2 is -T-L^{B2}-X

wherein X is a ligand comprising a saccharide, which may be fucose, mannose, or glucose; and L^{B2} is a covalent bond or a group derived from the covalent conjugation of a T with an X; and,

wherein n is 1, 2, or 3.

In particular embodiments, insulin oligosaccharide conjugate comprises an insulin oligosaccharide conjugate selected from the group consisting of IOC-1, 1OC-2, IOC-3, IOC-4, IOC-5, IOC-6, IOC-7, IOC-8, IOC-9, IOC-10, IOC-11, IOC-12, IOC-13, IOC-14, IOC-15, IOC-16, IOC-17, IOC-18, IOC-19, IOC-20, IOC-21, IOC-22, IOC-23, IOC-24, IOC-25, IOC-26, IOC-27, IOC-28, IOC-29, IOC-30, IOC-31, IOC-32, IOC-33, IOC-34, IOC-35, IOC-36, IOC-37, IOC-38, IOC-39, IOC-41, IOC-42, IOC-43, IOC-44, IOC-45, IOC-46, IOC-47, IOC-49, IOC-50, IOC-51, IOC-52, IOC-53, IOC-54, IOC-55, IOC-56, IOC-57, IOC-58, IOC-59, IOC-60, IOC-61, IOC-62, IOC-63, IOC-64, IOC-65, IOC-66, IOC-67, IOC-68, IOC-69, IOC-70, IOC-71, IOC-72, IOC-73, IOC-74, IOC-75, IOC-76, IOC-77, IOC-78, IOC-79, IOC-80, IOC-81, IOC-82, IOC-83, IOC-84, IOC-85, IOC-86, IOC-87, IOC-88, IOC-89, IOC-90, IOC-91, IOC-92, IOC-93, IOC-94, IOC-95, IOC-96, IOC-97, IOC-98, IOC-99, IOC-100, IOC-101, 1OC-102, IOC-103, IOC-104, IOC-105, IOC-106, IOC-107, IOC-108, IOC-109, IOC-110, IOC-111, IOC-112, IOC-113, IOC-114, IOC-115, IOC-116, IOC-117, IOC-118, IOC-119, IOC-120, IOC-121, IOC-122, IOC-123, IOC-124, IOC-125, IOC-126, IOC-127, IOC-128, IOC-129, IOC-130, IOC-131, IOC-132, IOC-133, IOC-134, IOC-135, IOC-136, IOC-137, IOC-138, IOC-139, IOC-140, IOC-141, IOC-142, IOC-143, IOC-144, IOC-145, IOC-146, IOC-147, IOC-149, IOC-150, IOC-151, IOC-152, IOC-153, IOC-154, IOC-155, IOC-156, IOC-157, IOC-158, IOC-159, IOC-160, IOC-161, IOC-162, IOC-163, IOC-164, IOC-165, IOC-166, IOC-167, IOC-168, IOC-169, IOC-170, IOC-171, IOC-172, IOC-173, IOC-174, IOC-175, IOC-176, IOC-177, IOC-178, IOC-179, IOC-180, IOC-181, IOC-182, IOC-183, IOC-184, IOC-185, IOC-186, IOC-187, IOC-188, IOC-189, IOC-190, IOC-191, IOC-192, IOC-193, 1OC-194, IOC-195, IOC-196, IOC-197, IOC-198, IOC-199, IOC-200, IOC-201, IOC-202, IOC-203, IOC-204, IOC-205, IOC-206, IOC-207, IOC-208, IOC-210, IOC-211, IOC-212, IOC-213, IOC-214, IOC-215, IOC-216, IOC-217, IOC-218, IOC-219, IOC-220, IOC-221, IOC-222, IOC-223, IOC-224, IOC-225, IOC-226, IOC-227, IOC-228, IOC-229, IOC-230, IOC-231, IOC-232, IOC-233, IOC-234, IOC-235, IOC-236, IOC-237, IOC-238, IOC-239, IOC-240, IOC-241, IOC-242, IOC-243, IOC-244, IOC-245, IOC-246, IOC-247, IOC-248, IOC-249, IOC-250, IOC-251, IOC-252, IOC-253, IOC-254, IOC-255, IOC-256, IOC-257, IOC-258, IOC-259, IOC-260, IOC-261, IOC-262, IOC-263, IOC-264, IOC-265, IOC-266, IOC-267, IOC-268, IOC-269, IOC-270, IOC-271, and IOC-272.

In particular embodiments, the insulin oligosaccharide conjugate comprises an insulin oligosaccharide conjugate having the structure

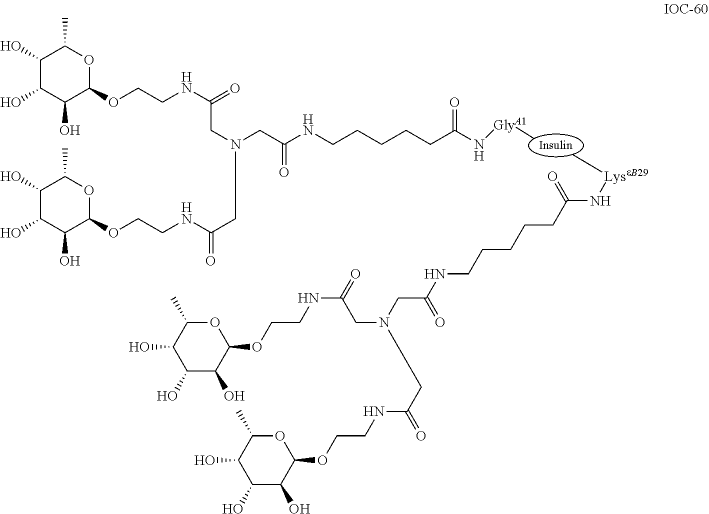

wherein the insulin is recombinant human insulin.

The present invention provides a method for controlling post-prandial glucose levels in an individual, comprising administering to the individual the formulation disclose herein at a particular time before a meal to control the post-prandial glucose levels in the individual. The present invention provides a treating an individual having diabetes comprising administering to the individual the formulation disclosed herein to treat the diabetes.

In particular embodiments, the diabetes comprises Diabetes type I, Diabetes type II, or gestational diabetes.

The present invention provides a pharmaceutical formulation disclosed herein for controlling post-prandial glucose levels in the individual.

The present invention provides a pharmaceutical formulation disclosed herein for treatment of diabetes.

In particular embodiments, the diabetes comprises Diabetes type I, Diabetes type II, or gestational diabetes, or gestational diabetes.

The present invention provides for use of a pharmaceutical formulation disclosed herein for manufacture of a medicament for controlling post-prandial glucose levels in the individual.

The present invention provides for use of a pharmaceutical formulation disclosed herein for manufacture of a medicament for treatment of diabetes.

In particular embodiments, the diabetes comprises Diabetes type I, Diabetes type II, or gestational diabetes.

### Definitions

Definitions of specific functional groups, chemical terms, and general terms used throughout the specification are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987.

Acyl—As used herein, the term “acyl,” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R—), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl; cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl, substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

Aliphatic—As used herein, the term “aliphatic” or “aliphatic group” denotes an optionally substituted hydrocarbon moiety that may be straight-chain (i.e., unbranched), branched, or cyclic (“carbocyclic”) and may be completely saturated or may contain one or more units of unsaturation, but which is not aromatic. Unless otherwise specified, aliphatic groups contain 1-12 carbon atoms. In some embodiments, aliphatic groups contain 1-6 carbon atoms. In some embodiments, aliphatic groups contain 1-4 carbon atoms, and in yet other embodiments aliphatic groups contain 1-3 carbon atoms. Suitable aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

Alkenyl—As used herein, the term “alkenyl” denotes an optionally substituted monovalent group derived from a straight- or branched-chain aliphatic moiety having at least one carbon-carbon double bond by the removal of a single hydrogen atom. In particular embodiments, the alkenyl group employed in the invention contains 2-6 carbon atoms. In particular embodiments, the alkenyl group employed in the invention contains 2-5 carbon atoms. In some embodiments, the alkenyl group employed in the invention contains 2-4 carbon atoms. In another embodiment, the alkenyl group employed contains 2-3 carbon atoms. Alkenyl groups include, for example, ethenyl, propenyl, butenyl, 1-methyl-2-buten-1-yl, and the like.

Alkyl—As used herein, the term “alkyl” refers to optionally substituted saturated, straight- or branched-chain hydrocarbon radicals derived from an aliphatic moiety containing between 1-6 carbon atoms by removal of a single hydrogen atom. In some embodiments, the alkyl group employed in the invention contains 1-5 carbon atoms. In another embodiment, the alkyl group employed contains 1-4 carbon atoms. In still other embodiments, the alkyl group contains 1-3 carbon atoms. In yet another embodiment, the alkyl group contains 1-2 carbons. Examples of alkyl radicals include, but are not limited to, methyl, ethyl, n-propyl, isopropyl, n-butyl, iso-butyl, sec-butyl, sec-pentyl, iso-pentyl, tert-butyl, n-pentyl, neopentyl, n-hexyl, sec-hexyl, n-heptyl, n-octyl, n-decyl, n-undecyl, dodecyl, and the like.

Alkynyl—As used herein, the term “alkynyl” refers to an optionally substituted monovalent group derived from a straight- or branched-chain aliphatic moiety having at least one carbon-carbon triple bond by the removal of a single hydrogen atom. In particular embodiments, the alkynyl group employed in the invention contains 2-6 carbon atoms. In particular embodiments, the alkynyl group employed in the invention contains 2-5 carbon atoms. In some embodiments, the alkynyl group employed in the invention contains 2-4 carbon atoms. In another embodiment, the alkynyl group employed contains 2-3 carbon atoms. Representative alkynyl groups include, but are not limited to, ethynyl, 2-propynyl (propargyl), 1-propynyl, and the like.

Aryl—As used herein, the term “aryl” used alone or as part of a larger moiety as in “aralkyl”, “aralkoxy”, or “aryloxyalkyl”, refers to an optionally substituted monocyclic and bicyclic ring systems having a total of five to 10 ring members, wherein at least one ring in the system is aromatic and wherein each ring in the system contains three to seven ring members. The term “aryl” may be used interchangeably with the term “aryl ring”. In particular embodiments of the present invention, “aryl” refers to an aromatic ring system which includes, but not limited to, phenyl, biphenyl, naphthyl, anthracyl and the like, which may bear one or more substituents.

Arylalkyl—As used herein, the term “arylalkyl” refers to an alkyl group substituted with an aryl group (e.g., an aromatic or heteroaromatic group).

Bidentate—a molecule formed from two or more molecules covalently bound together as a single unitmolecule.

Bivalent hydrocarbon chain—As used herein, the term “bivalent hydrocarbon chain” (also referred to as a “bivalent alkylene group”) is a polymethylene group, i.e., —(CH₂)_{z}—, wherein z is a positive integer from 1 to 30, from 1 to 20, from 1 to 12, from 1 to 8, from 1 to 6, from 1 to 4, from 1 to 3, from 1 to 2, from 2 to 30, from 2 to 20, from 2 to 10, from 2 to 8, from 2 to 6, from 2 to 4, or from 2 to 3. A substituted bivalent hydrocarbon chain is a polymethylene group in which one or more methylene hydrogen atoms are replaced with a substituent. Suitable substituents include those described below for a substituted aliphatic group.

Carbonyl—As used herein, the term “carbonyl” refers to a monovalent or bivalent moiety containing a carbon-oxygen double bond. Non-limiting examples of carbonyl groups include aldehydes, ketones, carboxylic acids, ester, amide, enones, acyl halides, anhydrides, ureas, carbamates, carbonates, thioesters, lactones, lactams, hydroxamates, isocyanates, and chloroformates.

Cycloaliphatic—As used herein, the terms “cycloaliphatic”, “carbocycle”, or “carbocyclic”, used alone or as part of a larger moiety, refer to an optionally substituted saturated or partially unsaturated cyclic aliphatic monocyclic or bicyclic ring systems, as described herein, having from 3 to 10 members. Cycloaliphatic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, and cyclooctadienyl. In some embodiments, the cycloalkyl has 3-6 carbons.

Fucose—refers to the D or L form of fucose and may refer to an oxygen or carbon linked glycoside.

Halogen—As used herein, the terms “halo” and “halogen” refer to an atom selected from fluorine (fluoro, —F), chlorine (chloro, —Cl), bromine (bromo, —Br), and iodine (iodo, —I).

Heteroaliphatic—As used herein, the terms “heteroaliphatic” or “heteroaliphatic group”, denote an optionally substituted hydrocarbon moiety having, in addition to carbon atoms, from one to five heteroatoms, that may be straight-chain (i.e., unbranched), branched, or cyclic (“heterocyclic”) and may be completely saturated or may contain one or more units of unsaturation, but which is not aromatic. Unless otherwise specified, heteroaliphatic groups contain 1-6 carbon atoms wherein 1-3 carbon atoms are optionally and independently replaced with heteroatoms selected from oxygen, nitrogen and sulfur. In some embodiments, heteroaliphatic groups contain 1-4 carbon atoms, wherein 1-2 carbon atoms are optionally and independently replaced with heteroatoms selected from oxygen, nitrogen and sulfur. In yet other embodiments, heteroaliphatic groups contain 1-3 carbon atoms, wherein 1 carbon atom is optionally and independently replaced with a heteroatom selected from oxygen, nitrogen and sulfur. Suitable heteroaliphatic groups include, but are not limited to, linear or branched, heteroalkyl, heteroalkenyl, and heteroalkynyl groups.

Heteroaralkyl—As used herein, the term “heteroaralkyl” refers to an alkyl group substituted by a heteroaryl, wherein the alkyl and heteroaryl portions independently are optionally substituted.

Heteroaryl—As used herein, the term “heteroaryl” used alone or as part of a larger moiety, e.g., “heteroaralkyl”, or “heteroaralkoxy”, refers to an optionally substituted group having 5 to 10 ring atoms, preferably 5, 6, or 9 ring atoms; having 6, 10, or 14 it electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, carbocyclic, or heterocyclic rings, where the radical or point of attachment is on the heteroaromatic ring. Non limiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, and tetrahydroisoquinolinyl. A heteroaryl group may be mono- or bicyclic. The term “heteroaryl” may be used interchangeably with the terms “heteroaryl ring”, “heteroaryl group”, or “heteroaromatic”, any of which terms include rings that are optionally substituted.

Heteroatom—As used herein, the term “heteroatom” refers to nitrogen, oxygen, or sulfur, and includes any oxidized form of nitrogen or sulfur, and any quaternized form of a basic nitrogen. The term “nitrogen” also includes a substituted nitrogen.

Heterocyclic—As used herein, the terms “heterocycle”, “heterocyclyl”, “heterocyclic radical”, and “heterocyclic ring” are used interchangeably and refer to a stable optionally substituted 5- to 7-membered monocyclic or 7- to 10-membered bicyclic heterocyclic moiety that is either saturated or partially unsaturated, and having, in addition to carbon atoms, one or more heteroatoms, as defined above. A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, pyrrolidinyl, pyrrolidonyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or carbocyclic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

Unsaturated—As used herein, the term “unsaturated”, means that a moiety has one or more double or triple bonds.

Partially unsaturated—As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

Optionally substituted—As described herein, compounds of the invention may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable”, as used herein, refers to compounds that are not substantially altered when subjected to conditions to allow for their production, detection, and, in particular embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph, which may be substituted with R^{∘}; —CH═CHPh, which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R)C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR)R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, or a 5- to 6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3- to 12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5- to 6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5- to 6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5- to 6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5- to 6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5- to 6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-to 12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5- to 6-membered saturated, partially unsaturated, or aryl ring having 0-4 heteroatoms independently selected from nitrogen, oxygen, or sulfur.

In any case where a chemical variable (e.g., an R group) is shown attached to a bond that crosses a bond of the ring, this means that one or more such variables are optionally attached to the ring having the crossed bond. Each R group on such a ring can be attached at any suitable position on the ring, this is generally understood to mean that the group is attached in place of a hydrogen atom on the parent ring. This includes the possibility that two R groups can be attached to the same ring atom. Furthermore, when more than one R group is present on a ring, each may be the same or different than other R groups attached thereto, and each group is defined independently of other groups that may be attached elsewhere on the same molecule, even though they may be represented by the same identifier.

Insulin or insulin molecule—the term is a generic term that designates the 51 amino acid heterodimer comprising the A-chain peptide having the amino acid sequence shown in SEQ ID NO: 1 and the B-chain peptide having the amino acid sequence shown in SEQ ID NO: 2, wherein the cysteine residues a positions 6 and 11 of the A chain are linked in a disulfide bond, the cysteine residues at position 7 of the A chain and position 7 of the B chain are linked in a disulfide bond, and the cysteine residues at position 20 of the A chain and 19 of the B chain are linked in a disulfide bond.

Insulin analog or analogue—the term as used herein includes any heterodimer analogue or single-chain analogue that comprises one or more modification(s) of the native A-chain peptide and/or B-chain peptide. Modifications include but are not limited to substituting an amino acid for the native amino acid at a position selected from A4, A5, A8, A9, A10, A12, A13, A14, A15, A16, A17, A18, A19, A21, B1, B2, B3, B4, B5, B9, B10, B13, B14, B15, B16, B17, B18, B20, B21, B22, B23, B26, B27, B28, B29, and B30; deleting any or all of positions B1-4 and B26-30; or conjugating directly or by a polymeric or non-polymeric linker one or more acyl, polyethylglycine (PEG), or saccharide moiety (moieties); or any combination thereof. As exemplified by the N-linked glycosylated insulin analogues disclosed herein, the term further includes any insulin heterodimer and single-chain analogue that has been modified to have at least one N-linked glycosylation site and in particular, embodiments in which the N-linked glycosylation site is linked to or occupied by an N-glycan. Examples of insulin analogues include but are not limited to the heterodimer and single-chain analogues disclosed in published international application WO20100080606, WO2009/099763, and WO2010080609, the disclosures of which are incorporated herein by reference. Examples of single-chain insulin analogues also include but are not limited to those disclosed in published International Applications WO9634882, WO95516708, WO2005054291, WO2006097521, WO2007104734, WO2007104736, WO2007104737, WO2007104738, WO2007096332, WO2009132129; U.S. Pat. Nos. 5,304,473 and 6,630,348; and Kristensen et al., Biochem. J. 305: 981-986 (1995), the disclosures of which are each incorporated herein by reference.

Treat—As used herein, the term “treat” (or “treating”, “treated”, “treatment”, etc.) refers to the administration of a conjugate of the present disclosure to a subject in need thereof with the purpose to alleviate, relieve, alter, ameliorate, improve or affect a condition (e.g., diabetes), a symptom or symptoms of a condition (e.g., hyperglycemia), or the predisposition toward a condition. For example, as used herein the term “treating diabetes” will refer in general to maintaining glucose blood levels near normal levels and may include increasing or decreasing blood glucose levels depending on a given situation.

Parenteral administration—As used herein, a parental route of administration means introducing a drug into the body through injection, for quicker absorption by the body. The injection may be intravenous, intramuscular, or subcutaneous.

Other embodiments, aspects and features of the present invention are either further described in or will be apparent from the ensuing description, examples and appended claims.

### BRIEF DESCRIPTION OF THE DRAWINGS

FIG. 1A. Tertiary structure of IOC-60 (monitored by near-UV-CD) as a function of buffer species and pH range. Formulation in this study is 4 mg/mL IOC-60, 0.5 molar equivalent zinc, 10 mM Tris buffer, 16 mg/mL glycerin, at the various pH values as labeled in the figure.

FIG. 1B. Tertiary structure of IOC-60 (monitored by near-UV-CD) as a function of buffer species and pH range. Formulation in this study is 4 mg/mL IOC-60, 0.5 molar equivalent zinc, 10 mM histidine buffer, 16 mg/mL glycerin, at the various pH values as labeled in the figure.

FIG. 1C. Tertiary structure of IOC-60 (monitored by near-UV-CD) as a function of buffer species and pH range. Formulation in this study is 4 mg/mL IOC-60, 0.5 molar equivalent zinc, 10 mM phosphate buffer, 16 mg/mL glycerin, at the various pH values as labeled in the figure.

FIG. 2. Nonlinear curve fitting of hexamer content (main %) determined by analytical ultracentrifugation as a function of Zn/IOC-60 monomer molar ratios with the data in Table 1.

FIG. 3A. Comparison of insulin glargine vs. formulations B0, B1, and B2. The dose was 0.6 nmol/kg for insulin glargine and 2.8 nmol/kg for IOC-60 formulations to account for the potency difference of the compounds.

FIG. 3B. Comparison of insulin glargine vs. formulation P4. The dose was 0.21 nmol/kg for insulin glargine and 2.8 nmol/kg for IOC-60 formulations to account for the potency difference of the compounds.

### DETAILED DESCRIPTION OF THE INVENTION

The present invention provides a pharmaceutical formulation comprising an insulin or insulin analog molecule covalently attached to at least one branched linker having a first arm and second arm, wherein the first arm is linked to a first ligand that includes a first saccharide and the second arm is linked to a second ligand that includes a second saccharide and wherein the first saccharide is fucose (herein referred to as insulin oligosaccharide conjugate; IOC), which is suitable for controlling post-prandial glucose levels in an individual.

The pharmaceutical formulation comprises or consists essentially of about 4 mg/mL to 40 mg/mL of an insulin oligosaccharide conjugate, 0.35 to 1.0 molar equivalent zinc (Zn²⁺) to the insulin oligosaccharide conjugate monomer, 5 to 50 mM sodium (Na) phosphate buffer, 5 to 100 mM halide, 16.0 mg/mL (174 mM) glycerin, 2.8 mg/mL (26 mM) phenolic compound at pH 6.2 to pH 7.0.

In particular embodiments the pharmaceutical formulation comprises or consists essentially of 4 mg/mL insulin oligosaccharide conjugate, 0.45 molar equivalent zinc to insulin oligosaccharide conjugate monomer (165 mM Zn²⁺), 7 mM sodium phosphate buffer, 15 mM halide, 16.0 mg/mL (174 mM) glycerin, 2.8 mg/mL (26 mM) phenolic compound, pH 6.6. The insulin oligosaccharide conjugate in this formulation is primarily in a hexameric form (about 80% of the insulin oligosaccharide conjugate is in hexamer form).

In a further embodiment, a pharmaceutical formulation comprising or consisting essentially of an insulin oligosaccharide conjugate suitable for IV administration is provided. The formulation comprises or consists essentially of 4.0 mg/mL insulin oligosaccharide conjugate, 0.0165 mg/mL zinc, 0.683 mg/mL sodium phosphate monobasic dihydrate, 0.373 mg/mL sodium phosphate dibasic anhydrous, 0.877 mg/mL halide, 16.0 mg/mL (174 mM) glycerin, 2.8 mg/mL (26 mM) phenolic compound, pH 6.6. The insulin oligosaccharide conjugate in this formulation is primarily in the hexamer form (greater than 90% hexamer form).

Phosphate buffer was identified as being capable of effectively maintaining protein tertiary structure and buffering capacity within the pH range between 6.2 and 7.0. A zinc to insulin oligosaccharide conjugate molar ratio within a defined range of 0.35 to 1.0 provides greater than 80% hexamer formation. The zinc may be provided as zinc acetate or zinc chloride. The zinc may be provided as zinc acetate or zinc chloride. In a particular embodiment, the zinc salt is zinc chloride (ZnCl₂).

The halide in the formulation may enhance hexamer formation. U.S. Pat. No. 5,866,538 discloses insulin formulations comprising a halide. In particular embodiments of the pharmaceutical formulation, the halide is sodium chloride (NaCl).

The phenolic compound may be used as a preservative and may comprise m-cresol or phenol.

In particular embodiments the pharmaceutical formulation comprises or consists essentially of 4 mg/mL insulin oligosaccharide conjugate, 0.45 molar equivalent zinc to insulin oligosaccharide conjugate monomer (165 mM Zn²⁺), 7 mM sodium phosphate buffer, 15 mM sodium chloride, 16.0 mg/mL (174 mM) glycerin, 2.8 mg/mL (26 mM) m-cresol at pH 6.6. The insulin oligosaccharide conjugate in this formulation is primarily in a hexameric form (about 80% of the insulin oligosaccharide conjugate is in hexamer form).

In a further embodiment, a pharmaceutical formulation comprising or consisting essentially of an insulin oligosaccharide conjugate suitable for intravenous (IV) administration is provided. The formulation comprises or consists essentially of 4.0 mg/mL insulin oligosaccharide conjugate, 0.0165 mg/mL zinc, 0.683 mg/mL (4.4 mM) sodium phosphate monobasic dehydrate and 0.373 mg/mL (2.6 mM) Na phosphate dibasic anhydrous to provide 7 mM sodium phosphate buffer, 0.877 mg/mL NaCl, 16.0 mg/mL (174 mM) glycerin, 2.8 mg/mL (26 mM) m-cresol, pH 6.6. The insulin oligosaccharide conjugate in this formulation is primarily in the hexamer form (greater than 90% hexamer form).

### Insulin Oligosaccharide Conjugates

In general, the insulin oligosaccharide conjugates comprise an insulin or insulin analog molecule covalently attached to at least one branched linker having or consisting of two arms, each arm independently covalently attached to a ligand comprising or consisting of a saccharide wherein at least one ligand of the linker includes the saccharide fucose. In particular embodiments, the ligands are capable of competing with a saccharide (e.g., glucose or alpha-methylmannose) for binding to an endogenous saccharide-binding molecule. In particular embodiments, the ligands are capable of competing with glucose or alpha-methylmannose for binding to Con A. In particular embodiments, the linker is non-polymeric. In particular embodiments, the conjugate may have a polydispersity index of one and a MW of less than about 20,000 Da. In particular embodiments, the conjugate is of formula (I) or (II) as defined and described herein. In particular embodiments, the conjugate is long acting (i.e., exhibits a PK profile that is more sustained than soluble recombinant human insulin (RHI)).

In one aspect, the present invention provides insulin oligosaccharide conjugates that comprise an insulin or insulin analog molecule covalently attached to at least one branched linker having two arms (bi-dentate linker) wherein each arm of the bi-dentate linker is independently covalently linked to a ligand comprising or consisting of a saccharide and wherein the first ligand of the bi-dentate linker comprises or consists of a first saccharide, which is fucose. The second ligand of the bi-dentate linker comprises or consists of a second saccharide, which may be fucose, mannose, glucosamine, or glucose. In particular aspects, the second ligand comprises or consists of a bisaccharide, trisaccharide, tetrasaccharide, or branched trisaccharide. In particular aspects, the second ligand comprises a bimannose, trimannose, tetramannose, or branched trimannose.

In particular aspects, the insulin or insulin analog molecule is conjugated to one, two, three, or four bi-dentate linkers wherein each arm of each bi-dentate linker is independently covalently linked to a ligand comprising or consisting of a saccharide and wherein the first ligand of the bi-dentate linker comprises or consists of a first saccharide, which is fucose, and the second ligand of the bi-dentate linker comprises or consists of a second saccharide, which may be fucose, mannose, or glucose. In particular aspects, the second ligand comprises or consists of a bisaccharide, trisaccharide, tetrasaccharide, or branched trisaccharide. In particular aspects, the second ligand comprises or consists of a bimannose, trimannose, tetramannose, or branched trimannose.

In particular aspects, the insulin or insulin analog molecule is conjugated to one, two, three, or four bi-dentate linkers wherein each arm of each bi-dentate linker is independently covalently linked to a ligand comprising or consisting of a saccharide and wherein for at least one of the bi-dentate linkers the first ligand of the bi-dentate linker comprises or consists of a first saccharide, which is fucose, and the second ligand of the bi-dentate linker comprises or consists of a second saccharide, which may be fucose, mannose, or glucose. In particular aspects, the second ligand comprises or consists of a bisaccharide, trisaccharide, tetrasaccharide, or branched trisaccharide. In particular aspects, the second ligand comprises or consists of a bimannose, trimannose, tetramannose, or branched trimannose. For the second, third, and fourth bi-dentate linkers, the first and second saccharides may independently be fucose, mannose, glucose, bisaccharide, trisaccharide, tetrasaccharide, branched trisaccharide, bimannose, trimannose, tetramannose, or branched trimannose.

In particular aspects, the insulin or insulin analog molecule is conjugated to (i) one bi-dentate linker wherein each arm of each bi-dentate linker is independently covalently linked to a ligand comprising or consisting of a saccharide wherein the first ligand of the bi-dentate linker comprises or consists of a first saccharide, which is fucose, and the second ligand of the bi-dentate linker comprises or consists of a second saccharide, which may be fucose, mannose, glucose, bisaccharide, trisaccharide, tetrasaccharide, branched trisaccharide, bimannose, trimannose, tetramannose, or branched trimannose.

In particular aspects, the insulin or insulin analog molecule of the insulin oligosaccharide conjugate disclosed herein is further covalently attached to at least one linear linker having one ligand comprising or consisting of a saccharide, which may be fucose, mannose, glucosamine, or glucose. In particular aspects, the ligand comprises or consisting of a bisaccharide, trisaccharide, tetrasaccharide, or branched trisaccharide. In particular aspects, the ligand comprises or consisting of a bimannose, trimannose, tetramannose, or branched trimannose.

In particular aspects, the insulin or insulin analog molecule conjugate disclosed herein is further covalently attached to at least one tri-dentate linker wherein each arm of the tri-dentate linker is independently covalently linked to a ligand comprising or consisting of a saccharide, which may be fucose, mannose, glucosamine, or glucose. In particular aspects, the ligand comprises or consisting of a bisaccharide, trisaccharide, tetrasaccharide, or branched trisaccharide. In particular aspects, the ligand comprises or consisting of a bimannose, trimannose, tetramannose, or branched trimannose.

The present invention provides pharmaceutical formulations wherein the insulin oligosaccharide conjugates comprising fucose display glucose responsiveness, i.e., a pharmacokinetic (PK) and/or pharmacodynamic (PD) profile that is responsive to the systemic concentrations of a saccharide such as glucose or alpha-methylmannose when administered to a subject in need thereof in the absence of an exogenous multivalent saccharide-binding molecule such as Con A. In further aspects, the conjugate binds an endogenous saccharide binding molecule at a serum glucose concentration of 60 or 70 mg/dL or less when administered to a subject in need thereof. The binding of the conjugate to the endogenous saccharide binding molecule is sensitive to the serum concentration of the serum saccharide. In a further aspect, the conjugate is capable of binding the insulin receptor at a serum saccharide concentration great than 60, 70, 80, 90, or 100 mg/dL. At serum saccharide concentration at 60 or 70 mg/dL the conjugate preferentially binds the endogenous saccharide binding molecule over the insulin receptor and as the serum concentration of the serum saccharide increases from 60 or 70 mg/dL, the binding of the conjugate to the endogenous saccharide binding molecule decreases and the binding of the conjugate to the insulin receptor increases.

In general, the conjugates comprise an insulin or insulin analog molecule covalently attached to at least one branched linker having two arms (bi-dentate linker), each arm independently attached to a ligand comprising a saccharide wherein at least one ligand of the linker is fucose. In particular embodiments, the linker is non-polymeric. In particular embodiments, a conjugate may have a polydispersity index of one and a MW of less than about 20,000 Da.

### Ligand(s)

In general, the insulin oligosaccharide conjugates comprise an insulin or insulin analog molecule covalently attached to at least one bi-dentate linker having two ligands wherein at least one of the ligands (the first ligand) comprises or consists of a saccharide, which is fucose, and the other ligand (the second ligand) comprises or consists of one or more saccharides. In particular embodiments, the insulin oligosaccharide conjugates may further include one or more linear linkers, each comprising a single ligand, which comprises or consist of one or more saccharides. In particular embodiments, the insulin oligosaccharide conjugates may further include one or more branched linkers that each includes at least two, three, four, five, or more ligands, where each ligand independently comprises or consists of one or more saccharides. When more than one ligand is present the ligands may have the same or different chemical structures.

In particular embodiments, the ligands are capable of competing with a saccharide (e.g., glucose, alpha-methylmannose, or mannose) for binding to an endogenous saccharide-binding molecule (e.g., without limitation surfactant proteins A and D or members of the selectin family). In particular embodiments, the ligands are capable of competing with a saccharide (e.g., glucose, alpha-methylmannose, or mannose) for binding to cell-surface sugar receptor (e.g., without limitation macrophage mannose receptor, glucose transporter ligands, endothelial cell sugar receptors, or hepatocyte sugar receptors). In particular embodiments, the ligands are capable of competing with glucose for binding to an endogenous glucose-binding molecule (e.g., without limitation surfactant proteins A and D or members of the selectin family). In particular embodiments, the ligands are capable of competing with glucose or alpha-mewthylmannose for binding to the human macrophage mannose receptor 1 (MRC1). In particular embodiments, the ligands are capable of competing with a saccharide for binding to a non-human lectin (e.g., Con A). In particular embodiments, the ligands are capable of competing with glucose, alpha-methylmannose, or mannose for binding to a non-human lectin (e.g., Con A). Exemplary glucose-binding lectins include calnexin, calreticulin, N-acetylglucosamine receptor, selectin, asialoglycoprotein receptor, collectin (mannose-binding lectin), mannose receptor, aggrecan, versican, pisum sativum agglutinin (PSA), vicia faba lectin, lens culinaris lectin, soybean lectin, peanut lectin, lathyrus ochrus lectin, sainfoin lectin, sophorajaponica lectin, bowringia milbraedii lectin, concanavalin A (Con A), and pokeweed mitogen.

In particular embodiments, the ligand(s) other than the first ligand comprising or consisting of the saccharide fucose may have the same chemical structure as glucose or may be a chemically related species of glucose, e.g., glucosamine. In various embodiments, it may be advantageous for the ligand(s) to have a different chemical structure from glucose, e.g., in order to fine tune the glucose response of the conjugate. For example, in particular embodiments, one might use a ligand that includes glucose, mannose, L-fucose or derivatives of these (e.g., alpha-L-fucopyranoside, mannosamine, beta-linked N-acetyl mannosamine, methylglucose, methylmannose, ethylglucose, ethylmannose, propylglucose, propylmannose, etc.) and/or higher order combinations of these (e.g., a bimannose, linear and/or branched trimannose, etc.).

In particular embodiments, the ligand(s) include(s) a monosaccharide. In particular embodiments, the ligand(s) include(s) a disaccharide. In particular embodiments, the ligand(s) include(s) a trisaccharide. In some embodiments, the ligand(s) comprise a saccharide and one or more amine groups. In some embodiments, the ligand(s) comprise a saccharide and ethyl group. In particular embodiments, the saccharide and amine group are separated by a C₁-C₆ alkyl group, e.g., a C₁-C₃ alkyl group. In some embodiments, the ligand is aminoethylglucose (AEG). In some embodiments, the ligand is aminoethylmannose (AEM). In some embodiments, the ligand is aminoethylbimannose (AEBM). In some embodiments, the ligand is aminoethyltrimannose (AETM). In some embodiments, the ligand is β-aminoethyl-N-acetylglucosamine (AEGA). In some embodiments, the ligand is aminoethylfucose (AEF). In particular embodiments, the saccharide is of the “d” configuration and in other embodiments, the saccharide is of the “1” configuration. Below are the structures of exemplary saccharides having an amine group separated from the saccharide by a C₂ ethyl group wherein R may be hydrogen or a carbonyl group of the linker. Other exemplary ligands will be recognized by those skilled in the art.

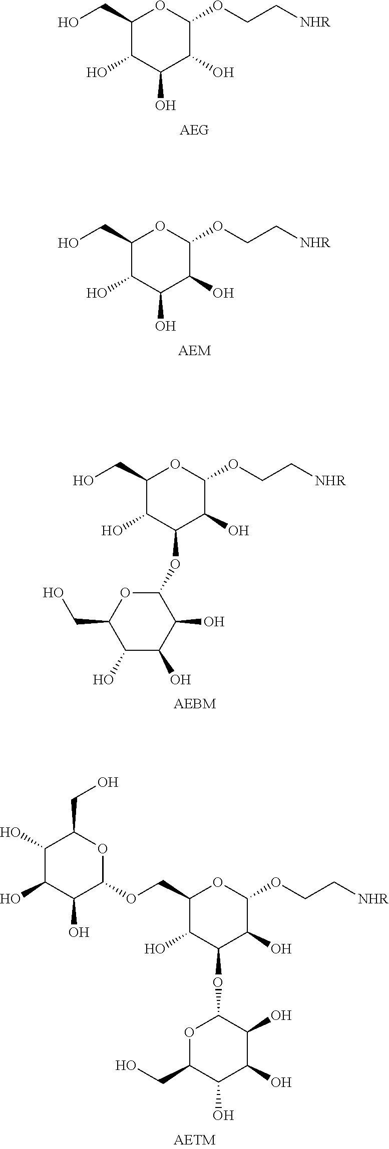

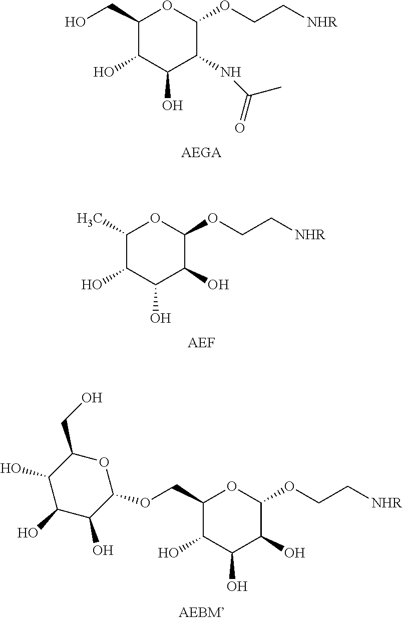

### Insulin

As used herein, the term “insulin” or “insulin molecule” encompasses all salt and non-salt forms of the insulin molecule. It will be appreciated that the salt form may be anionic or cationic depending on the insulin molecule. By “insulin” or “an insulin molecule” we intend to encompass both wild-type insulin and modified forms of insulin as long as they are bioactive (i.e., capable of causing a detectable reduction in glucose when administered in vivo). Wild-type insulin includes insulin from any species whether in purified, synthetic or recombinant form (e.g., human insulin, porcine insulin, bovine insulin, rabbit insulin, sheep insulin, etc.). A number of these are available commercially, e.g., from Sigma-Aldrich (St. Louis, Mo.). A variety of modified forms of insulin are known in the art (e.g. see Crotty and Reynolds, Pediatr. Emerg. Care. 23:903-905, 2007 and Gerich, Am. J. Med. 113:308-16, 2002 and references cited therein). Modified forms of insulin (insulin analogs) may be chemically modified (e.g., by addition of a chemical moiety such as a PEG group or a fatty acyl chain as described below) and/or mutated (i.e., by addition, deletion or substitution of one or more amino acids). In particular embodiments, an insulin molecule of the present disclosure will differ from a wild-type insulin by 1-10 (e.g., 1-9, 1-8, 1-7, 1-6, 1-5, 1-4, 1-3, 1-2, 2-9, 2-8, 2-7, 2-6, 2-5, 2-4, 2-3, 3-9, 3-8, 3-7, 3-6, 3-5, 3-4, 4-9, 4-8, 4-7, 4-6, 4-5, 5-9, 5-8, 5-7, 5-6, 6-9, 6-8, 6-7, 7-9, 7-8, 8-9, 9, 8, 7, 6, 5, 4, 3, 2 or 1) amino acid substitutions, additions and/or deletions. In particular embodiments, an insulin molecule of the present disclosure will differ from a wild-type insulin by amino acid substitutions only. In particular embodiments, an insulin molecule of the present disclosure will differ from a wild-type insulin by amino acid additions only. In particular embodiments, an insulin molecule of the present disclosure will differ from wild-type insulin by both amino acid substitutions and additions. In particular embodiments, an insulin molecule of the present disclosure will differ from a wild-type insulin by both amino acid substitutions and deletions.

In particular embodiments, amino acid substitutions may be made on the basis of similarity in polarity, charge, solubility, hydrophobicity, hydrophilicity, and/or the amphipathic nature of the residues involved. In particular embodiments, a substitution may be conservative, that is, one amino acid is replaced with one of similar shape and charge. Conservative substitutions are well known in the art and typically include substitutions within the following groups: glycine, alanine; valine, isoleucine, leucine; aspartic acid, glutamic acid; asparagine, glutamine; serine, threonine; lysine, arginine; and tyrosine, phenylalanine. In particular embodiments, the hydrophobic index of amino acids may be considered in choosing suitable mutations. The importance of the hydrophobic amino acid index in conferring interactive biological function on a polypeptide is generally understood in the art. Alternatively, the substitution of like amino acids can be made effectively on the basis of hydrophilicity. The importance of hydrophilicity in conferring interactive biological function of a polypeptide is generally understood in the art. The use of the hydrophobic index or hydrophilicity in designing polypeptides is further discussed in U.S. Pat. No. 5,691,198.

The wild-type sequence of human insulin (A-chain and B-chain) is shown below.

A-Chain (SEQ ID NO: 1):
GIVEQCCTSICSLYQLENYCN
B-Chain (SEQ ID NO: 2):
FVNQHLCGSHLVEALYLVCGERGFFYTPKT

In various embodiments, an insulin molecule of the present disclosure is mutated at the B28 and/or B29 positions of the B-peptide sequence. For example, insulin lispro (HUMALOG®) is a rapid acting insulin mutant in which the penultimate lysine and proline residues on the C-terminal end of the B-peptide have been reversed (LysB28ProB29-human insulin) (SEQ ID NO:3). This modification blocks the formation of insulin multimers. Insulin aspart (NOVOLOG®) is another rapid acting insulin mutant in which proline at position B28 has been substituted with aspartic acid (AspB28-human insulin) (SEQ ID NO:4). This mutant also prevents the formation of multimers. In some embodiments, mutation at positions B28 and/or B29 is accompanied by one or more mutations elsewhere in the insulin polypeptide. For example, insulin glulisine (APIDRA®) is yet another rapid acting insulin mutant in which aspartic acid at position B3 has been replaced by a lysine residue and lysine at position B29 has been replaced with a glutamic acid residue (LysB3GluB29-human insulin) (SEQ ID NO:5).

In various embodiments, an insulin molecule of the present disclosure has an isoelectric point that is shifted relative to human insulin. In some embodiments, the shift in isoelectric point is achieved by adding one or more arginine residues to the N-terminus of the insulin A-peptide and/or the C-terminus of the insulin B-peptide. Examples of such insulin polypeptides include ArgA0-human insulin, ArgB31ArgB32-human insulin, GlyA21ArgB31ArgB32-human insulin, ArgA0ArgB31ArgB32-human insulin, and ArgA0GlyA21ArgB31ArgB32-human insulin. By way of further example, insulin glargine (LANTUS®) is an exemplary long acting insulin mutant in which AspA21 has been replaced by glycine (SEQ ID NO:6), and two arginine residues have been added to the C-terminus of the B-peptide (SEQ ID NO:7). The effect of these changes is to shift the isoelectric point, producing a solution that is completely soluble at pH 4. Thus, in some embodiments, an insulin molecule of the present disclosure comprises an A-peptide sequence wherein A21 is Gly and B-peptide sequence wherein B31 and B32 are Arg-Arg. It is to be understood that the present disclosure encompasses all single and multiple combinations of these mutations and any other mutations that are described herein (e.g., GlyA21-human insulin, GlyA21ArgB31-human insulin, ArgB31ArgB32-human insulin, ArgB31-human insulin).

In various embodiments, an insulin molecule of the present disclosure is truncated. For example, in particular embodiments, a B-peptide sequence of an insulin polypeptide of the present disclosure is missing B1, B2, B3, B26, B27, B28, B29 and/or B30. In particular embodiments, combinations of residues are missing from the B-peptide sequence of an insulin polypeptide of the present disclosure. For example, the B-peptide sequence may be missing residues B(1-2), B(1-3), B(29-30), B(28-30), B(27-30) and/or B(26-30). In some embodiments, these deletions and/or truncations apply to any of the aforementioned insulin molecules (e.g., without limitation to produce des(B30)-insulin lispro, des(B30)-insulin aspart, des(B30)-insulin glulisine, des(B30)-insulin glargine, etc.).

### Exemplary Insulin Carbohydrate Conjugates

In various embodiments, the insulin oligosaccharide conjugate of the present disclosure comprises an insulin or insulin analog molecule conjugated to at least one bi-dentate linker wherein at least one arm of the bi-dentate linker is attached to the ligand aminoethylfucose (AEF). The other arm of the bi-dentate linker may be conjugated to the ligand AEF and/or one or more ligands that are independently selected from the group consisting of aminoethylglucose (AEG), aminoethylmannose (AEM), aminoethylbimannose (AEBM), aminoethyltrimannose (AETM), β-aminoethyl-N-acetylglucosamine (AEGA), and aminoethylfucose (AEF). In particular embodiments, the insulin molecule is conjugated via the A1 amino acid residue. In particular embodiments, the insulin molecule is conjugated via the B1 amino acid residue. In particular embodiments, the insulin molecule is conjugated via the epsilon-amino group of LysB29. In particular embodiments, the insulin molecule is an analog that comprises a lysine at position B28 (LysB28), and the insulin molecule is conjugated via the epsilon-amino group of LysB28, for example, insulin lispro conjugated via the epsilon-amino group of LysB28. In particular embodiments, the insulin molecule is an analog that comprises a lysine at position B3 (LysB3), and the insulin molecule is conjugated via the epsilon-amino group of LysB3, for example, insulin glulisine conjugated via the epsilon-amino group of LysB3.

In particular embodiments, the insulin or insulin molecule of the above insulin oligosaccharide conjugate may be conjugated to one or more additional linkers attached to one or more ligands, each ligand independently selected from aminoethylglucose (AEG), aminoethylmannose (AEM), aminoethylbimannose (AEBM) ligands, aminoethyltrimannose (AETM) ligands, β-aminoethyl-N-acetylglucosamine (AEGA), and aminoethylfucose (AEF). The additional linkers may be linear, bi-dentate, tri-dentate, quadri-dentate, etc. wherein each arm of the linker comprises a ligand that may indenpendently be selected from aminoethylglucose (AEG), aminoethylmannose (AEM), aminoethylbimannose (AEBM) ligands, aminoethyltrimannose (AETM) ligands, β-aminoethyl-N-acetylglucosamine (AEGA), and aminoethylfucose (AEF).

Thus, in particular embodiments, the insulin oligosaccharide conjugate may comprise or consist of a bi-dentate linker wherein at least one arm of the bi-dentate linker is attached to the ligand aminoethylfucose (AEF) conjugated to the amino group at position A1 of the insulin or insulin analog; or the amino group at position B1 of the insulin or insulin analog; or the amino group at position B3 of the insulin analog; or the amino group at position B28 of the insulin analog; or the amino group at position B29 of the insulin or insulin analog.

In particular embodiments, the insulin oligosaccharide conjugate may comprise or consist of two bi-dentate linkers wherein a first bi-dentate linker, which has the ligand aminoethylfucose (AEF) attached to one arm of the first bi-dentate linker and a ligand selected from aminoethylglucose (AEG), aminoethylmannose (AEM), aminoethylbimannose (AEBM), aminoethyltrimannose (AETM), β-aminoethyl-N-acetylglucosamine (AEGA), and aminoethylfucose (AEF) attached to the other arm of the bi-dentate linker, is conjugated to the amino group at position A1 and a second bi-dentate linker attached to one or more ligands, each ligand independently selected from aminoethylglucose (AEG), aminoethylmannose (AEM), aminoethylbimannose (AEBM), aminoethyltrimannose (AETM), β-aminoethyl-N-acetylglucosamine (AEGA), and aminoethylfucose (AEF) is conjugated to the amino group at position B1, B3, B28, or B29.

In particular embodiments, the insulin oligosaccharide conjugate may comprise or consist of two bi-dentate linkers wherein a first bi-dentate linker, which has the ligand aminoethylfucose (AEF) attached to one arm of the first bi-dentate linker and a ligand selected from aminoethylglucose (AEG), aminoethylmannose (AEM), aminoethylbimannose (AEBM), aminoethyltrimannose (AETM), β-aminoethyl-N-acetylglucosamine (AEGA), and aminoethylfucose (AEF) attached to the other arm of the bi-dentate linker, is conjugated to the amino group at position B1 and a second bi-dentate linker attached to one or more ligands, each ligand independently selected from aminoethylglucose (AEG), aminoethylmannose (AEM), aminoethylbimannose (AEBM), aminoethyltrimannose (AETM), β-aminoethyl-N-acetylglucosamine (AEGA), and aminoethylfucose (AEF) is conjugated to the amino group at position A1, B3, B28, or B29.

In particular embodiments, the insulin oligosaccharide conjugate may comprise or consist of two bi-dentate linkers wherein a first bi-dentate linker, which has the ligand aminoethylfucose (AEF) attached to one arm of the first bi-dentate linker and a ligand selected from aminoethylglucose (AEG), aminoethylmannose (AEM), aminoethylbimannose (AEBM), aminoethyltrimannose (AETM), β-aminoethyl-N-acetylglucosamine (AEGA), and aminoethylfucose (AEF) attached to the other arm of the bi-dentate linker, is conjugated to the amino group at position B3 and a second bi-dentate linker attached to one or more ligands, each ligand independently selected from aminoethylglucose (AEG), aminoethylmannose (AEM), aminoethylbimannose (AEBM), aminoethyltrimannose (AETM), β-aminoethyl-N-acetylglucosamine (AEGA), and aminoethylfucose (AEF) is conjugated to the amino group at position B1, A1, B28, or B29.

In particular embodiments, the insulin oligosaccharide conjugate may comprise or consist of two bi-dentate linkers wherein a first bi-dentate linker, which has the ligand aminoethylfucose (AEF) attached to one arm of the first bi-dentate linker and a ligand selected from aminoethylglucose (AEG), aminoethylmannose (AEM), aminoethylbimannose (AEBM), aminoethyltrimannose (AETM), β-aminoethyl-N-acetylglucosamine (AEGA), and aminoethylfucose (AEF) attached to the other arm of the bi-dentate linker, is conjugated to the amino group at position B28 and a second bi-dentate linker attached to one or more ligands, each ligand independently selected from aminoethylglucose (AEG), aminoethylmannose (AEM), aminoethylbimannose (AEBM), aminoethyltrimannose (AETM), β-aminoethyl-N-acetylglucosamine (AEGA), and aminoethylfucose (AEF) is conjugated to the amino group at position B1, B3, A1, or B29.

In particular embodiments, the insulin oligosaccharide conjugate may comprise or consist of two bi-dentate linkers wherein a first bi-dentate linker, which has the ligand aminoethylfucose (AEF) attached to one arm of the first bi-dentate linker and a ligand selected from aminoethylglucose (AEG), aminoethylmannose (AEM), aminoethylbimannose (AEBM), aminoethyltrimannose (AETM), β-aminoethyl-N-acetylglucosamine (AEGA), and aminoethylfucose (AEF) attached to the other arm of the bi-dentate linker, is conjugated to the amino group at position B29 and a second bi-dentate linker attached to one or more ligands, each ligand independently selected from aminoethylglucose (AEG), aminoethylmannose (AEM), aminoethylbimannose (AEBM), aminoethyltrimannose (AETM), β-aminoethyl-N-acetylglucosamine (AEGA), and aminoethylfucose (AEF) is conjugated to the amino group at position B1, B3, B28, or A1.

In particular embodiments, the insulin oligosaccharide conjugate may comprise or consist of three bi-dentate linkers wherein a first bi-dentate linker, which has the ligand aminoethylfucose (AEF) attached to one arm of the first bi-dentate linker and a ligand selected from aminoethylglucose (AEG), aminoethylmannose (AEM), aminoethylbimannose (AEBM), aminoethyltrimannose (AETM), β-aminoethyl-N-acetylglucosamine (AEGA), and aminoethylfucose (AEF) attached to the other arm of the bi-dentate linker, is conjugated to the amino group at position A1; a second bi-dentate linker attached to one or more ligands, each ligand independently selected from aminoethylglucose (AEG), aminoethylmannose (AEM), aminoethylbimannose (AEBM), aminoethyltrimannose (AETM), β-aminoethyl-N-acetylglucosamine (AEGA), and aminoethylfucose (AEF) is conjugated to the amino group at position B1; and, a third bi-dentate linker attached to one or more ligands, each ligand independently selected from aminoethylglucose (AEG), aminoethylmannose (AEM), aminoethylbimannose (AEBM), aminoethyltrimannose (AETM), β-aminoethyl-N-acetylglucosamine (AEGA), and aminoethylfucose (AEF) is conjugated to the amino group at position B3, B28, or B29.

In particular embodiments, the insulin oligosaccharide conjugate may comprise or consist of four bi-dentate linkers wherein a first bi-dentate linker, which has the ligand aminoethylfucose (AEF) attached to one arm of the first bi-dentate linker and a ligand selected from aminoethylglucose (AEG), aminoethylmannose (AEM), aminoethylbimannose (AEBM), aminoethyltrimannose (AETM), β-aminoethyl-N-acetylglucosamine (AEGA), and aminoethylfucose (AEF) attached to the other arm of the bi-dentate linker, is conjugated to the amino group at position A1; a second bi-dentate linker attached to one or more ligands, each ligand independently selected from aminoethylglucose (AEG), aminoethylmannose (AEM), aminoethylbimannose (AEBM), aminoethyltrimannose (AETM), β-aminoethyl-N-acetylglucosamine (AEGA), and aminoethylfucose (AEF) is conjugated to the amino group at position B1; a third bi-dentate linker attached to one or more ligands, each ligand independently selected from aminoethylglucose (AEG), aminoethylmannose (AEM), aminoethylbimannose (AEBM), aminoethyltrimannose (AETM), β-aminoethyl-N-acetylglucosamine (AEGA), and aminoethylfucose (AEF) is conjugated to the amino group at position B3; and a fourth bi-dentate linker attached to one or more ligands, each ligand independently selected from aminoethylglucose (AEG), aminoethylmannose (AEM), aminoethylbimannose (AEBM), aminoethyltrimannose (AETM), β-aminoethyl-N-acetylglucosamine (AEGA), and aminoethylfucose (AEF) is conjugated to the amino group at position B28 or B29.

In particular embodiments, the insulin oligosaccharide conjugate may comprise or consist of (a) a bi-dentate linker wherein at least one arm of the bi-dentate linker is attached to the ligand aminoethylfucose (AEF) conjugated to the amino group at position A1; or the amino group at position B1; or the amino group at position B3; or the amino group at position B28; or the amino group at position B29 and (b) a linear or tri-dentate linker attached to one or more ligands, each ligand independently selected from aminoethylglucose (AEG), aminoethylmannose (AEM), aminoethylbimannose (AEBM), aminoethyltrimannose (AETM), β-aminoethyl-N-acetylglucosamine (AEGA), and aminoethylfucose (AEF) conjugated to the amino group at position A1; or the amino group at position B1; or the amino group at position B3; or the amino group at position B28; or the amino group at position B29, whichever position is not occupied by the bi-dentate linker.

In particular embodiments, the insulin oligosaccharide conjugate may comprise or consist of (a) a bi-dentate linker, which has the ligand aminoethylfucose (AEF) attached to one arm of the first bi-dentate linker and a ligand selected from aminoethylglucose (AEG), aminoethylmannose (AEM), aminoethylbimannose (AEBM), aminoethyltrimannose (AETM), β-aminoethyl-N-acetylglucosamine (AEGA), and aminoethylfucose (AEF) attached to the other arm of the bi-dentate linker, conjugated to the amino group at position A1 and (b) a linear or tri-dentate linker attached to one or more ligands, each ligand independently selected from aminoethylglucose (AEG), aminoethylmannose (AEM), aminoethylbimannose (AEBM), aminoethyltrimannose (AETM), β-aminoethyl-N-acetylglucosamine (AEGA), and aminoethylfucose (AEF) conjugated to the amino group at position B1, B3, B28, or B29.

In particular embodiments, the insulin oligosaccharide conjugate may comprise or consist of (a) a bi-dentate linker, which has the ligand aminoethylfucose (AEF) attached to one arm of the first bi-dentate linker and a ligand selected from aminoethylglucose (AEG), aminoethylmannose (AEM), aminoethylbimannose (AEBM), aminoethyltrimannose (AETM), (3-aminoethyl-N-acetylglucosamine (AEGA), and aminoethylfucose (AEF) attached to the other arm of the bi-dentate linker, conjugated to the amino group at position B1 and (b) a linear or tri-dentate linker attached to one or more ligands, each ligand independently selected from aminoethylglucose (AEG), aminoethylmannose (AEM), aminoethylbimannose (AEBM), aminoethyltrimannose (AETM), j-aminoethyl-N-acetylglucosamine (AEGA), and aminoethylfucose (AEF) conjugated to the amino group at position A1, B3, B28, or B29.

In particular embodiments, the insulin oligosaccharide conjugate may comprise or consist of (a) a bi-dentate linker, which has the ligand aminoethylfucose (AEF) attached to one arm of the first bi-dentate linker and a ligand selected from aminoethylglucose (AEG), aminoethylmannose (AEM), aminoethylbimannose (AEBM), aminoethyltrimannose (AETM), β-aminoethyl-N-acetylglucosamine (AEGA), and aminoethylfucose (AEF) attached to the other arm of the bi-dentate linker, conjugated to the amino group at position B3 and (b) a linear or tri-dentate linker attached to one or more ligands, each ligand independently selected from aminoethylglucose (AEG), aminoethylmannose (AEM), aminoethylbimannose (AEBM), aminoethyltrimannose (AETM), β-aminoethyl-N-acetylglucosamine (AEGA), and aminoethylfucose (AEF) conjugated to the amino group at position B1, A1, B28, or B29.

In particular embodiments, the insulin oligosaccharide conjugate may comprise or consist of (a) a bi-dentate linker, which has the ligand aminoethylfucose (AEF) attached to one arm of the first bi-dentate linker and a ligand selected from aminoethylglucose (AEG), aminoethylmannose (AEM), aminoethylbimannose (AEBM), aminoethyltrimannose (AETM), β-aminoethyl-N-acetylglucosamine (AEGA), and aminoethylfucose (AEF) attached to the other arm of the bi-dentate linker, conjugated to the amino group at position B28 and (b) a linear or tri-dentate linker attached to one or more ligands, each ligand independently selected from aminoethylglucose (AEG), aminoethylmannose (AEM), aminoethylbimannose (AEBM), aminoethyltrimannose (AETM), β-aminoethyl-N-acetylglucosamine (AEGA), and aminoethylfucose (AEF) conjugated to the amino group at position B1, B3, A1, or B29.

In particular embodiments, the insulin oligosaccharide conjugate may comprise or consist of (a) a bi-dentate linker, which has the ligand aminoethylfucose (AEF) attached to one arm of the first bi-dentate linker and a ligand selected from aminoethylglucose (AEG), aminoethylmannose (AEM), aminoethylbimannose (AEBM), aminoethyltrimannose (AETM), 3-aminoethyl-N-acetylglucosamine (AEGA), and aminoethylfucose (AEF) attached to the other arm of the bi-dentate linker, conjugated to the amino group at position B29 and (b) a linear or tri-dentate linker attached to one or more ligands, each ligand independently selected from aminoethylglucose (AEG), aminoethylmannose (AEM), aminoethylbimannose (AEBM), aminoethyltrimannose (AETM), β-aminoethyl-N-acetylglucosamine (AEGA), and aminoethylfucose (AEF) conjugated to the amino group at position B1, B3, B28, or A1.

In particular embodiments, the insulin oligosaccharide conjugate may comprise or consist of (a) a bi-dentate linker, which has the ligand aminoethylfucose (AEF) attached to one arm of the first bi-dentate linker and a ligand selected from aminoethylglucose (AEG), aminoethylmannose (AEM), aminoethylbimannose (AEBM), aminoethyltrimannose (AETM), β-aminoethyl-N-acetylglucosamine (AEGA), and aminoethylfucose (AEF) attached to the other arm of the bi-dentate linker; (b) a first linear or tri-dentate linker attached to one or more ligands, each ligand independently selected from aminoethylglucose (AEG), aminoethylmannose (AEM), aminoethylbimannose (AEBM), aminoethyltrimannose (AETM), β-aminoethyl-N-acetylglucosamine (AEGA), and aminoethylfucose (AEF); and (c) a second linear or tri-dentate linker attached to one or more ligands, each ligand independently selected from aminoethylglucose (AEG), aminoethylmannose (AEM), aminoethylbimannose (AEBM), aminoethyltrimannose (AETM), β-aminoethyl-N-acetylglucosamine (AEGA), and aminoethylfucose (AEF), wherein each linker is each conjugated to an amino group at position A1, B1, B3, B28, or B29 with the proviso that each occupies a separate position such that three sites in total are occupied.

In various embodiments, the conjugates may have the general formula (I):

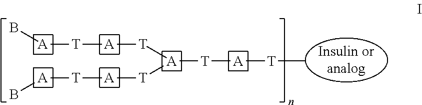

wherein:

each occurrence of

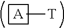

represents a potential repeat within a branch of the conjugate;

each occurrence of [symbol] is independently a covalent bond, a carbon atom, a heteroatom, or an optionally substituted group selected from the group consisting of acyl, aliphatic, heteroaliphatic, aryl, heteroaryl, and heterocyclic;

each occurrence of T is independently a covalent bond or a bivalent, straight or branched, saturated or unsaturated, optionally substituted C₁₋₃₀ hydrocarbon chain wherein one or more methylene units of T are optionally and independently replaced by —O—, —S—, —N(R)—, —C(O)—, —C(O)O—, —OC(O)—, —N(R)C(O)—, —C(O)N(R)—, —S(O)—, —S(O)₂—, —N(R)SO₂—, —SO₂N(R)—, a heterocyclic group, an aryl group, or a heteroaryl group;

each occurrence of R is independently hydrogen, a suitable protecting group, or an acyl moiety, arylalkyl moiety, aliphatic moiety, aryl moiety, heteroaryl moiety, or heteroaliphatic moiety;

—B is -T-L^{B}-X; each occurrence of X is independently a ligand;

each occurrence of L^{B} is independently a covalent bond or a group derived from the covalent conjugation of a T with an X; and,

wherein n is 1, 2, or 3, with the proviso that the insulin is conjugated to at least one linker in which one of the ligands is fucose.

In particular embodiments, the insulin or insulin analog conjugate may have the general formula (II):

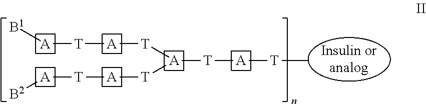

wherein:

each occurrence of

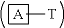

represents a potential repeat within a branch of the conjugate;

each occurrence of [symbol] is independently a covalent bond, a carbon atom, a heteroatom, or an optionally substituted group selected from the group consisting of acyl, aliphatic, heteroaliphatic, aryl, heteroaryl, and heterocyclic;

each occurrence of T is independently a covalent bond or a bivalent, straight or branched, saturated or unsaturated, optionally substituted C₁₋₃₀ hydrocarbon chain wherein one or more methylene units of T are optionally and independently replaced by —O—, —S—, —N(R)—, —C(O)—, —C(O)O—, —OC(O)—, —N(R)C(O)—, —C(O)N(R)—, —S(O)—, —S(O)₂—, —N(R)SO₂—, —SO₂N(R)—, a heterocyclic group, an aryl group, or a heteroaryl group;

each occurrence of R is independently hydrogen, a suitable protecting group, or an acyl moiety, arylalkyl moiety, aliphatic moiety, aryl moiety, heteroaryl moiety, or heteroaliphatic moiety;

—B1 is -T-L^{B1}-fucose;

wherein L^{B1} is a covalent bond or a group derived from the covalent conjugation of a T with an X;

—B2 is -T-L^{B2}-X;

wherein X is a ligand comprising a saccharide, which may be fucose, mannose, or glucose; and L^{B2} is a covalent bond or a group derived from the covalent conjugation of a T with an X; and,

wherein n is 1, 2, or 3.

Exemplary human insulin oligosaccharide conjugates (IOCs) include the IOCs disclosed in U.S. Pat. No. 9,427,475, which is incorporated herein by reference in its entirety. In particular, the following IOCs disclosed in U.S. Pat. No. 9,427,475 from column 89 through column 384, that is IOC-1, 1OC-2, IOC-3, IOC-4, IOC-5, IOC-6, IOC-7, IOC-8, IOC-9, IOC-10, IOC-11, IOC-12, IOC-13, IOC-14, IOC-15, IOC-16, IOC-17, IOC-18, IOC-19, IOC-20, IOC-21, IOC-22, IOC-23, IOC-24, IOC-25, IOC-26, IOC-27, IOC-28, IOC-29, IOC-30, IOC-31, IOC-32, IOC-33, IOC-34, IOC-35, IOC-36, IOC-37, IOC-38, IOC-39, IOC-41, IOC-42, IOC-43, IOC-44, IOC-45, IOC-46, IOC-47, IOC-49, IOC-50, IOC-51, IOC-52, IOC-53, IOC-54, IOC-55, IOC-56, IOC-57, IOC-58, IOC-59, IOC-60, IOC-61, IOC-62, IOC-63, IOC-64, IOC-65, IOC-66, IOC-67, IOC-68, IOC-69, IOC-70, IOC-71, IOC-72, IOC-73, IOC-74, IOC-75, IOC-76, IOC-77, IOC-78, IOC-79, IOC-80, IOC-81, IOC-82, IOC-83, IOC-84, IOC-85, IOC-86, IOC-87, IOC-88, IOC-89, IOC-90, IOC-91, IOC-92, IOC-93, IOC-94, IOC-95, IOC-96, IOC-97, IOC-98, IOC-99, IOC-100, IOC-101, 10OC-102, IOC-103, IOC-104, IOC-105, IOC-106, IOC-107, IOC-108, IOC-109, IOC-110, IOC-111, IOC-112, IOC-113, IOC-114, IOC-115, IOC-116, IOC-117, IOC-118, IOC-119, IOC-120, IOC-121, IOC-122, IOC-123, IOC-124, IOC-125, IOC-126, IOC-127, IOC-128, IOC-129, IOC-130, IOC-131, IOC-132, IOC-133, IOC-134, IOC-135, IOC-136, IOC-137, IOC-138, IOC-139, IOC-140, IOC-141, IOC-142, IOC-143, IOC-144, IOC-145, IOC-146, IOC-147, IOC-149, IOC-150, IOC-151, IOC-152, IOC-153, IOC-154, IOC-155, IOC-156, IOC-157, IOC-158, IOC-159, IOC-160, IOC-161, IOC-162, IOC-163, IOC-164, IOC-165, IOC-166, IOC-167, IOC-168, IOC-169, IOC-170, IOC-171, IOC-172, IOC-173, IOC-174, IOC-175, IOC-176, IOC-177, IOC-178, IOC-179, IOC-180, IOC-181, IOC-182, IOC-183, IOC-184, IOC-185, IOC-186, IOC-187, IOC-188, IOC-189, IOC-190, IOC-191, IOC-192, IOC-193, 1OC-194, IOC-195, IOC-196, IOC-197, IOC-198, IOC-199, IOC-200, IOC-201, IOC-202, IOC-203, IOC-204, IOC-205, IOC-206, IOC-207, IOC-208, IOC-210, IOC-211, IOC-212, IOC-213, IOC-214, IOC-215, IOC-216, IOC-217, IOC-218, IOC-219, IOC-220, IOC-221, IOC-222, IOC-223, IOC-224, IOC-225, IOC-226, IOC-227, IOC-228, IOC-229, IOC-230, IOC-231, IOC-232, IOC-233, IOC-234, IOC-235, IOC-236, IOC-237, IOC-238, IOC-239, IOC-240, IOC-241, IOC-242, IOC-243, IOC-244, IOC-245, IOC-246, IOC-247, IOC-248, IOC-249, IOC-250, IOC-251, IOC-252, IOC-253, IOC-254, IOC-255, IOC-256, IOC-257, IOC-258, IOC-259, IOC-260, IOC-261, IOC-262, IOC-263, IOC-264, IOC-265, IOC-266, IOC-267, IOC-268, IOC-269, IOC-270, IOC-271, and IOC-272 are all incorporated herein by reference.

IOC-60 was exemplified in the Examples herein. IOC-60, is a semi-synthetic insulin saccharide conjugate with two identical fucose-containing branched linkers at the α-GlyA1 and ε-LysB29 positions, i.e., N2,1A,N6,29B-Bis[6-(2-{bis[2-({2-[(6-deoxy-α-L-galactopyranosyl)oxy]ethyl}amino)-2-oxoethyl]amino}acetamido) hexanoyl]human insulin. IOC-60 has an isoelectric point of 4.7 and has the structure

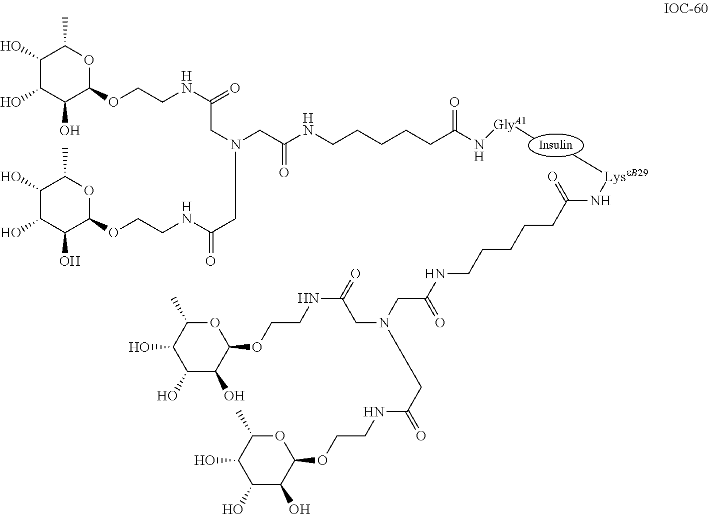

wherein the insulin is human recombinant insulin comprising the wild-type human insulin A chain polypeptide and B chain polypeptide. IOC-60 is synthesized from RHI and its sugar-linker precursor with ca. 50% yield (based on RHI). The synthesis of IOC-60 as well as the other IOCs mentioned above is described in U.S. Pat. No. 9,427,475, the methods of which are incorporated herein by reference.

### Uses of Pharmaceutical Formulations

In another aspect, the present disclosure provides methods of using the pharmaceutical formulations comprising the insulin oligosaccharide conjugate. In general, the pharmaceutical formulations comprising the insulin oligosaccharide conjugate can be used to controllably provide insulin to an individual in need in response to a saccharide (e.g., glucose or an exogenous saccharide such as mannose, alpha-methyl mannose, L-fucose, etc.). The disclosure encompasses treating diabetes by administering a pharmaceutical formulation comprising the insulin oligosaccharide conjugate. Although the insulin oligosaccharide conjugates can be used to treat any patient (e.g., dogs, cats, cows, horses, sheep, pigs, mice, etc.), they are most preferably used in the treatment of humans. A pharmaceutical formulation comprising the insulin oligosaccharide conjugate may be administered to a patient by any route. In general, the present disclosure encompasses administration intravenously or subcutaneously.

In general, a therapeutically effective amount of the pharmaceutical formulation comprising the insulin oligosaccharide conjugate will be administered. The term “therapeutically effective amount” means a sufficient amount of the pharmaceutical formulation comprising the insulin oligosaccharide conjugate to treat diabetes at a reasonable benefit/risk ratio, which involves a balancing of the efficacy and toxicity of the insulin oligosaccharide conjugate. In various embodiments, the average daily dose of insulin is in the range of 10 to 200 U, e.g., 25 to 100 U (where 1 Unit of insulin is ˜0.04 mg). In particular embodiments, the pharmaceutical formulation comprising the insulin oligosaccharide conjugate with these insulin doses is administered on a daily basis.

In particular embodiments, a pharmaceutical formulation comprising the insulin oligosaccharide conjugate may be used to treat hyperglycemia in a patient (e.g., a mammalian or human patient). In particular embodiments, the patient is diabetic. However, the present methods are not limited to treating diabetic patients. For example, in particular embodiments, a conjugate may be used to treat hyperglycemia in a patient with an infection associated with impaired glycemic control. In particular embodiments, a conjugate may be used to treat diabetes.

In particular embodiments, when a pharmaceutical formulation comprising the insulin oligosaccharide conjugate is administered to a patient (e.g., a mammalian patient) it induces less a pharmaceutical formulation comprising the insulin oligosaccharide conjugate induces a lower HbA1c value in a patient (e.g., a mammalian or human patient) than a formulation comprising an unconjugated version of the insulin molecule. In particular embodiments, the formulation leads to an HbA1c value that is at least 10% lower (e.g., at least 20% lower, at least 30% lower, at least 40% lower, at least 50% lower) than a formulation comprising an unconjugated version of the insulin molecule. In particular embodiments, the formulation leads to an HbA1c value of less than 7%, e.g., in the range of about 4 to about 6%. In particular embodiments, a formulation comprising an unconjugated version of the insulin molecule leads to an HbA1c value in excess of 7%, e.g., about 8 to about 12%.

The following examples are intended to promote a further understanding of the present invention.

### General Methods

### Sedimentation Velocity-Analytical Ultracentrifugation (SV-AUC)

Samples subject to AUC analysis were run neat at a concentration of approximately ˜4 mg/mL. The AUC cells were prepared with AUC-Abs quartz windows and meniscus matching center pieces. A Beckman-Coulter ProteomeLab XL-I AUC was used to collect absorbance data at 280 nm at 20° C. Scans were performed every 4 minutes for a total of 600 scans per cell at a rotation speed of 60,000 RPM. Data was analyzed using the SEDFIT software (version 13.0b) in a c(s) distribution model. The relative percentage of each species was calculated by integration.

### Circular Dichroism (CD)

Near-UV-CD was run on an automated circular dichroism (ACD) instrument from Applied Photophysics from 350 to 250 nm. Far-UV-CD was run on the same instrument from 250 to 200 nm. Five scans were recorded and averaged for each sample using a data pitch and bandwidth of 1 nm, and time per point of 3 seconds. Cuvette with 1 cm path length was used for near-UV-CD while cuvette with 1 mm path length was used for far-UV-CD. The spectra from placebo run were subtracted from GRI samples. The obtained CD signal was normalized by protein concentration.

### Dynamic Light Scattering (DLS)

DLS was performed using a Zetasizer Nano ZS (Malvem Instruments, Worcestershire, UK) at an angle of 173o by utilizing a noninvasive backscatter technique. Samples were run at ˜4 mg/mL at 20° C. The autocorrelation function was obtained and intensity based size distributions were compared among various formulations.

### Example 1

This example compared three different buffer systems (Tris, Histidine, Phosphate) to determine the pH range within each buffer system that gives a constant tertiary structure (hexamer) for IOC-60.

Tertiary structure of IOC-60 (monitored by near-UV-CD) as a function of buffer species and pH range was determined for each buffer system: System 1a: 10 mM Tris buffer; System 1b: 10 mM histidine buffer; System 1c: 10 mM phosphate buffer.

Each formulation comprised 4 mg/mL IOC-60, 0.5 molar equivalent zinc, 10 mM buffer, 16 mg/mL glycerin, at various pH values. For the formulations comprising Tris buffer the pH was 7.85, 7.47, 7.07, 6.64, 6.15, or 5.51. For the formulations comprising histidine buffer the pH was 7.06, 6.57, 6.00, or 5.58. For the formulations comprising phosphate buffer the pH was 7.91, 7.40, 7.05, 6.73, 6.30, 6.10, or 5.41.

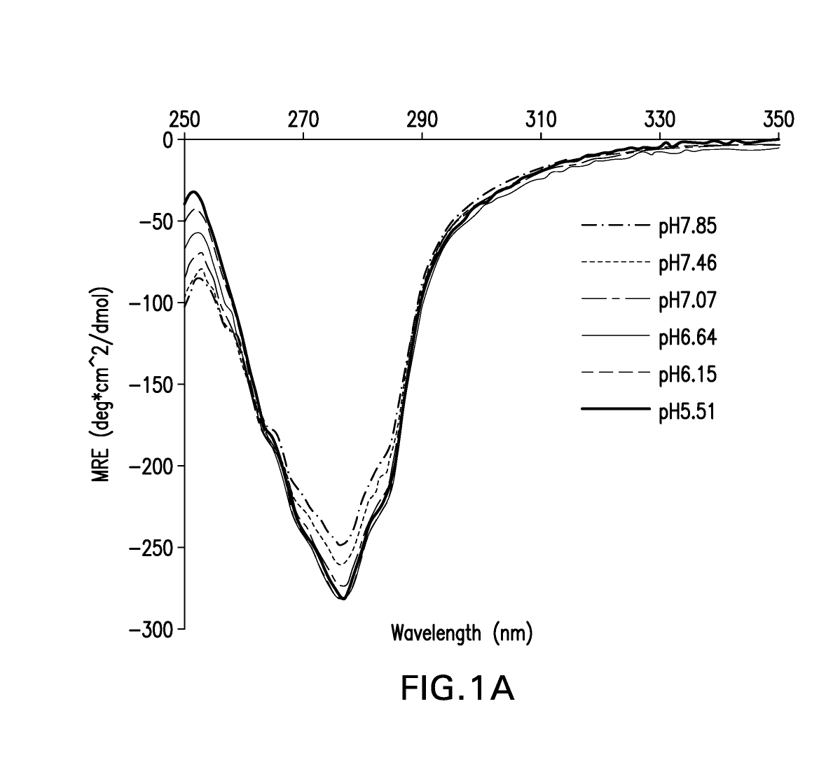

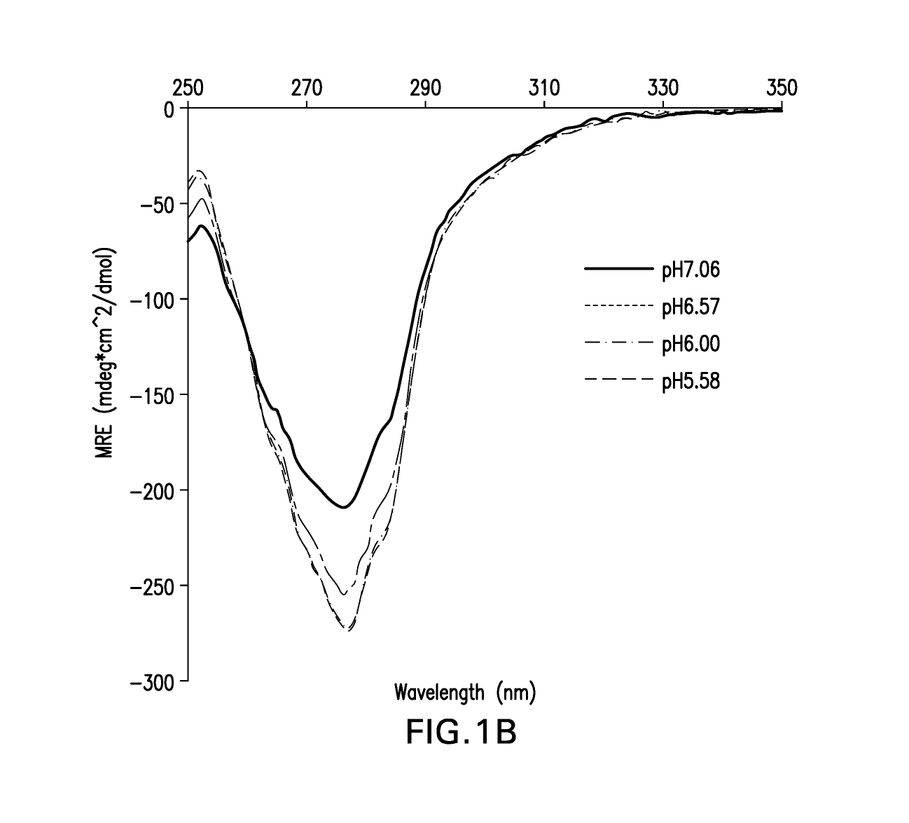

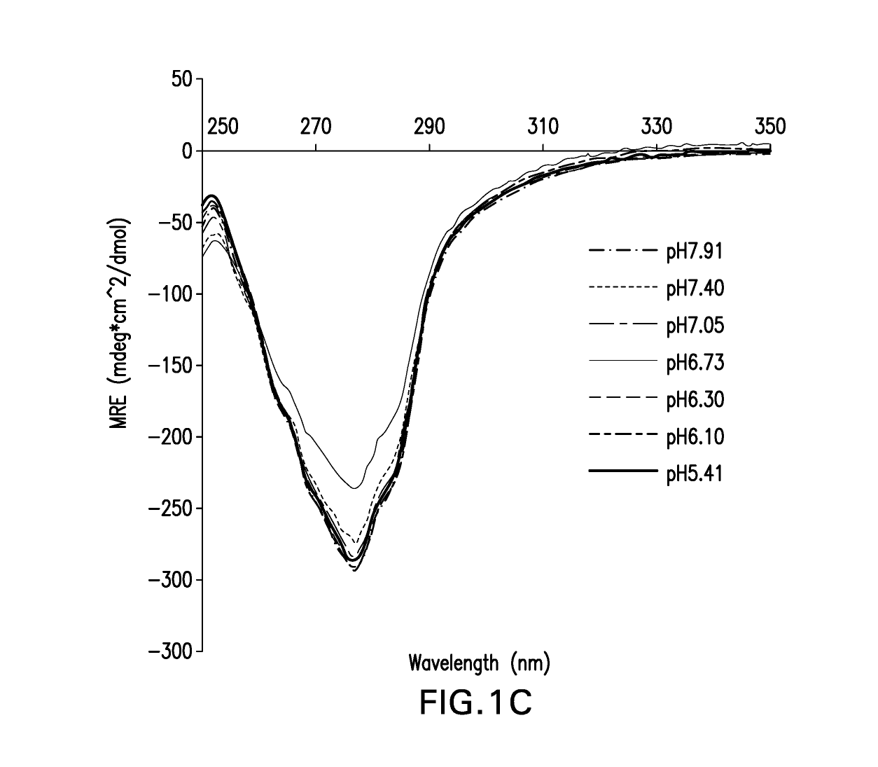

The results are shown in FIGS. 1A, 1B, and 1C. FIG. 1A shows that IOC-60 displayed constant tertiary structure in the pH range between 5.51 and 7.07 in 10 mM Tris buffer (pKa of Tris is 8.1, the buffering range is from 7.1 to 9.1). FIG. 1B shows that IOC-60 displayed constant tertiary structure in the pH range between 5.58 and 6.00 in 10 mM Histidine buffer (pKa of His is 6.0, the buffering range is from 5.0 to 7.0). FIG. 1C shows that IOC-60 displays constant tertiary structure in the pH range between 5.41 and 7.05 in 10 mM Phosphate buffer (pKa of Phos is 7.2, the buffering range is from 6.2 to 8.2).

### Selection of Phosphate Buffer and pH Range of 6.2 to 7.0 for IOC-60

Phosphate was selected as the buffer for IOC-60 formulation for the following reasons:

Tris has buffering capacity from about pH 7.1 to 9.1. IOC-60 starts to lose tertiary structure (hexamer form) above pH 7.07. Therefore, Tris does not have buffering capacity in the pH range in which IOC-60 has constant tertiary structure (i.e., pH 5.51 to 7.07).

Although IOC-60 shows constant tertiary structure in the pH range between 5.58 and 6.00 (within the buffering capacity of Histidine), this pH range is close to the isoelectric point (pI=5) of IOC-60 that may limit its solubility in the formulation. Also, use of Histidine buffer may compromise hexamer formation by outcompeting zinc binding to IOC-60 that is coordinated through histidine residuals in its sequence.

Phosphate buffer has buffering capacity within the pH range between 6.2 and 7.0, which is also the pH range that IOC-60 shows constant tertiary structure. The pH range (6.2 to 7.0) is defined by the overlap of pH range with constant tertiary structure (5.41 to 7.05) and pH range with buffering capacity (6.2 to 8.2). The overlapping pH range (6.2 to 7.0) in phosphate buffer was therefore selected for IOC-60 formulation. This pH range (6.2-7.0) and its midpoint (6.6) is the formulation pH range and target for IOC-60.

### Example 2

In this example the impact of Zinc hexamer formation of IOC-60 was determined.

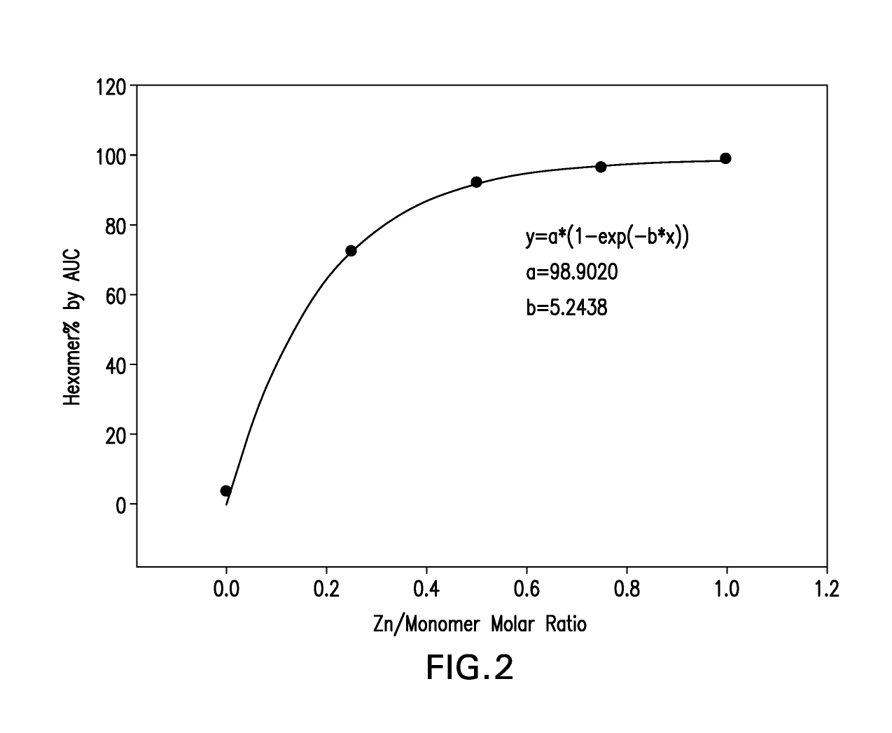

In the absence of Zinc, IOC-60 exists predominantly as monomer and dimer. However, the presence of Zinc dramatically favored hexamer formation with about 72% hexamer at a molar ratio of 0.25 while about 99% at a molar ratio of 1.0 (Table 1). A nonlinear curve fitting was performed to model the hexamer content determined by AUC as a function of Zn/IOC-60 molar ratio (FIG. 2), which showed a rapid rising phase from a molar ratio of 0.0 to 0.25 followed by a slow rising phase from 0.25 to 1.0. The curve predicts that greater than 80% hexamer can be achieved when the zinc/IOC-60 ratio is the range between 0.35 and 1.0. The curve also predicts that 90% hexamer can be achieved when the zinc/IOC-60 ratio is 0.45 (zinc target to be claimed in the formulation patent).

TABLE 1
Impact of Zn/IOC-60 molar ratio on hexamer formation
(by AUC).

| Zn/GRI Molar Ratio | Premain % | Main % | Postmain % |
|---|---|---|---|
| 0.00 | 96.4 | 3.6 | 0.0 |
| 0.25 | 27.4 | 72.2 | 0.4 |
| 0.50 | 7.5 | 92.0 | 0.6 |
| 0.75 | 2.1 | 96.3 | 1.7 |
| 1.00 | 0.9 | 98.8 | 0.2 |

Premain: low molecular weight species (monomer and dimer);
Main: Hexamer;
Postmain: Aggregates.
All values were experimentally determined by AUC.
Formulation in this study is 4 mg/mL IOC-60, 0 to 1 molar equivalent zinc, 10 mM phosphate, 16 mg/mL glycerin, pH 6.6.

### Example 3

This example shows that sodium chloride is beneficial to IOC-60 hexamer formation.

The impact of NaCl concentration on hexamer formation was studied by analytical ultracentrifugation (AUC). Compared to NaCl free formulation, there is a 3% increase of hexamer content in the presence of 20 mM NaCl (Table 2). Meanwhile, the average hydrodynamic diameter has increased from 5.8 to 6.7 nm in the presence of NaCl (as measured by dynamic light scattering) that further supports increase in hexamer formation. Further increase of NaCl concentration from 20 mM to 100 mM only slightly increased hexamer formation. 15 mM NaCl (target, range will be 5-100 mM) was actually chosen in IOC-60 formulation and tested in Ph I clinical trials.

TABLE 2
Effect of NaCl concentration on IOC-60 hexamer formation.

| | Area % | | |
|---|---|---|---|
| NaCl (mM) | Premain | Main | Postmain |
| 0 | 10.6 | 87.5 | 1.9 |
| 20 | 8.3 | 90.5 | 1.2 |
| 100 | 6.7 | 92.1 | 1.3 |

All values were experimentally determined by AUC.
Formulation in this study is 4 mg/ml IOC-60, 0.5 molar equivalent zinc, 0 to 100 mM NaCl, 10 mM phosphate, 16 mg/ml glycerin, pH 6.6.

### Example 4

Various formulations (Table 3) were tested in minipig models.

TABLE 3
Formulation description for various formulations

| | Formulation without PA | | | | Basal Formulation with PA | | | | |
|---|---|---|---|---|---|---|---|---|---|
| Components | B0 | B1 | B2 | P4 | B3 | B4 | B5 | B21 | B23 |
| IOC-60 (mg/Ml) | 4 | 12 | 12 | 20 | 20 | 20 | 40 | 20 | 40 |
| Zinc (mol. Eq.) | 0.45 | 0.45 | 0.45 | 0.45 | 0.45 | 0.45 | 0.45 | 0.45 | 0.45 |
| Protamine acetate (mg/mL) | — | — | — | — | 7 | 9 | 15 | 10 | 16 |
| Phosphate (mM) | 7 | 7 | — | — | 7 | — | — | 15 | 25 |
| m-cresol (mg/mL) | 2.8 | 2.8 | 2.8 | 2.8 | 2.8 | 2.8 | 2.8 | 2.8 | 2.8 |
| Glycerin (mg/mL) | 16.0 | 16.0 | 16.0 | 16.0 | 16.0 | 16.0 | 16.0 | 16.0 | 16.0 |
| NaCl (mM) | 15 | 15 | 15 | 15 | 15 | 15 | 15 | 15 | 15 |
| pH | 6.6 | 6.6 | 6.6 | 6.6 | 5.8 | 6.2 | 6.3 | 6.2 | 6.2 |

PA is protamine acetate

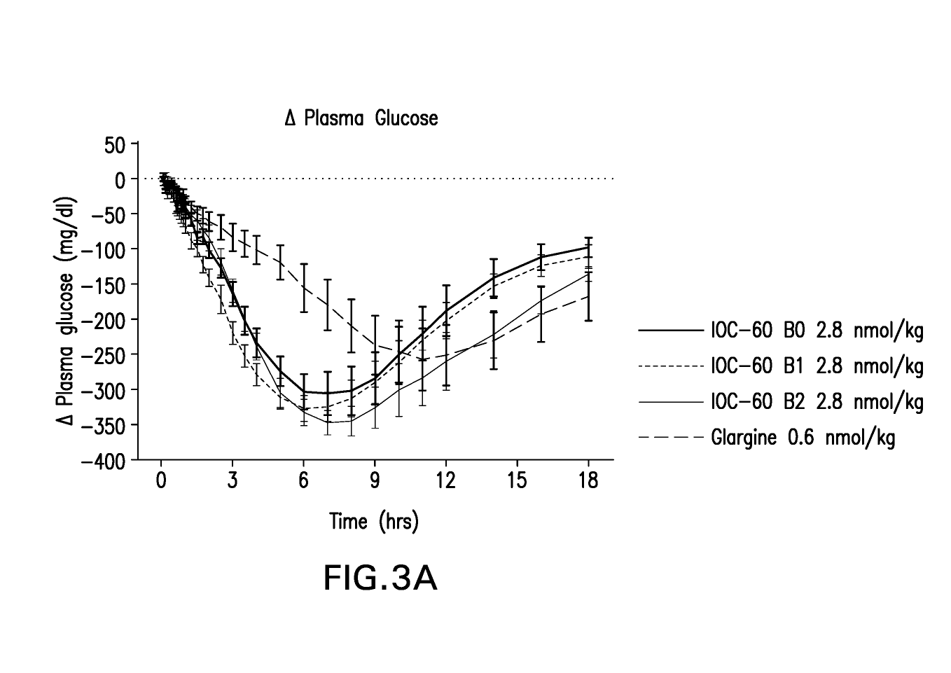

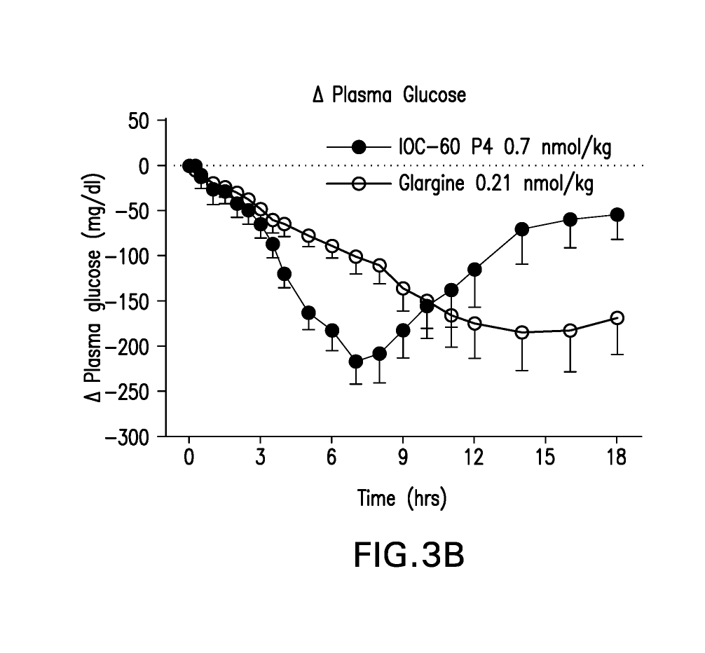

Each formulation was tested in eight animals in a crossover design. Blood glucose level was monitored for 18 hours. Insulin glargine (LANTUS, Sanofi) is a basal insulin and was used as a control for profile comparison. FIG. 3A shows a comparison of insulin glargine vs. B0, B1, B2, and B3 formulations. The dose was 0.6 nmol/kg for insulin glargine and 2.8 nmol/kg for IOC-60 formulations to account for the potency difference of the compounds. FIG. 3B shows a comparison of Glargine vs. P4. The dose was 0.21 nmol/kg for insulin glargine and 2.8 nmol/kg for IOC-60 formulations to account for the potency difference of the compounds.

It will be appreciated that various of the above-discussed and other features and functions, or alternatives thereof, may be desirably combined into many other different systems or applications. Also that various presently unforeseen or unanticipated alternatives, modifications, variations or improvements therein may be subsequently made by those skilled in the art which are also intended to be encompassed by the following claims.

1. A pharmaceutical formulation comprising an insulin oligosaccharide conjugate, sodium phosphate buffer, zinc salt, halide, glycerin, and phenolic compound,
wherein the formulation has a pH in the range of 6.2 to 7.0; wherein the insulin oligosaccharide conjugate comprises an insulin or insulin analog molecule covalently attached to at least one branched linker having a first arm and second arm, wherein the first arm is linked to a first ligand that includes a first saccharide and the second arm is linked to a second ligand that includes a second saccharide; and, wherein the first saccharide is fucose.

2. The pharmaceutical formulation of claim 1, wherein the formulation comprises about 4 mg/mL to 40 mg/mL insulin oligosaccharide conjugate, a molar equivalent zinc salt to the insulin oligosaccharide conjugate ratio of about 0.35 to 1.0 monomer, about 5 to 50 mM sodium phosphate buffer, about 5 to 100 mM halide, about 16.0 mg/mL glycerin, and about 2.8 mg/mL phenolic compound.

3. The pharmaceutical formulation of claim 1, wherein the formulation comprises about 4 mg/mL insulin oligosaccharide conjugate, a molar equivalent zinc salt to the insulin oligosaccharide conjugate monomer ratio of about 0.45, about 7 mM sodium phosphate buffer, about 15 mM halide, about 16.0 mg/mL glycerin, and about 2.8 mg/mL phenolic compound.

4. The pharmaceutical formulation of claim 1, wherein the zinc salt is selected from zinc acetate and zinc salt.

5. The pharmaceutical formulation of claim 1, wherein the halide is sodium chloride.

6. The pharmaceutical formulation of claim 1, wherein the phenolic compound is m-cresol.

7. A pharmaceutical formulation consisting essentially of an insulin oligosaccharide conjugate, sodium phosphate buffer, zinc salt, halide, glycerin, and phenolic compound,
wherein the formulation has a pH in the range of 6.2 to 7.0; wherein the insulin oligosaccharide conjugate comprises an insulin or insulin analog molecule covalently attached to at least one branched linker having a first arm and second arm, wherein the first arm is linked to a first ligand that includes a first saccharide and the second arm is linked to a second ligand that includes a second saccharide; and, wherein the first saccharide is fucose.

8. The pharmaceutical formulation of claim 7, wherein the formulation comprises about 4 mg/mL to 40 mg/mL insulin oligosaccharide conjugate, a molar equivalent zinc salt to the insulin oligosaccharide conjugate monomer ratio of about 0.35 to 1.0, about 5 to 50 mM sodium phosphate buffer, about 5 to 100 mM halide, about 16.0 mg/mL glycerin, and about 2.8 mg/mL phenolic compound.

9. The pharmaceutical formulation of claim 7, wherein the formulation comprises about 4 mg/mL insulin oligosaccharide conjugate, a molar equivalent zinc salt to the insulin oligosaccharide conjugate ratio of about 0.45, about 7 mM sodium phosphate buffer, about 15 mM halide, about 16.0 mg/mL glycerin, and about 2.8 mg/mL phenolic compound.

10. The pharmaceutical formulation of claim 7, wherein the zinc salt is selected from zinc acetate and zinc salt.

11. The pharmaceutical formulation of claim 7, wherein the halide is sodium chloride.

12. The pharmaceutical formulation of claim 7, wherein the phenolic compound is m-cresol.

13. The pharmaceutical formulation of claim 1, wherein the insulin oligosaccharide conjugate comprises general formula (I): 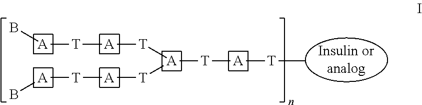
wherein:
each occurrence of 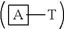
represents a potential repeat within a branch of the conjugate;
each occurrence of [symbol] is independently a covalent bond, a carbon atom, a heteroatom, or an optionally substituted group selected from the group consisting of acyl, aliphatic, heteroaliphatic, aryl, heteroaryl, and heterocyclic;
each occurrence of T is independently a covalent bond or a bivalent, straight or branched, saturated or unsaturated, optionally substituted C₁₋₃₀ hydrocarbon chain wherein one or more methylene units of T are optionally and independently replaced by —O—, —S—, —N(R)—, —C(O)—, —C(O)O—, —OC(O)—, —N(R)C(O)—, —C(O)N(R)—, —S(O)—, —S(O)₂—, —N(R)SO₂—, —SO₂N(R)—, a heterocyclic group, an aryl group, or a heteroaryl group;
each occurrence of R is independently hydrogen, a suitable protecting group, or an acyl moiety, arylalkyl moiety, aliphatic moiety, aryl moiety, heteroaryl moiety, or heteroaliphatic moiety;
—B is -T-L^{B}-X;
each occurrence of X is independently a ligand;
each occurrence of L^{B} is independently a covalent bond or a group derived from the covalent conjugation of a T with an X; and,
wherein n is 1, 2, or 3, with the proviso that the insulin is conjugated to at least one linker in which one of the ligands is fucose.

14. The pharmaceutical formulation of claim 1, wherein the insulin oligosaccharide conjugate comprises general formula (II): 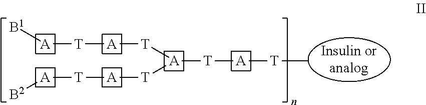
wherein:
each occurrence of 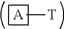
represents a potential repeat within a branch of the conjugate;
each occurrence of [symbol] is independently a covalent bond, a carbon atom, a heteroatom, or an optionally substituted group selected from the group consisting of acyl, aliphatic, heteroaliphatic, aryl, heteroaryl, and heterocyclic;
each occurrence of T is independently a covalent bond or a bivalent, straight or branched, saturated or unsaturated, optionally substituted C₁₋₃₀ hydrocarbon chain wherein one or more methylene units of T are optionally and independently replaced by —O—, —S—, —N(R)—, —C(O)—, —C(O)O—, —OC(O)—, —N(R)C(O)—, —C(O)N(R)—, —S(O)—, —S(O)₂—, —N(R)SO₂—, —SO₂N(R)—, a heterocyclic group, an aryl group, or a heteroaryl group;
each occurrence of R is independently hydrogen, a suitable protecting group, or an acyl moiety, arylalkyl moiety, aliphatic moiety, aryl moiety, heteroaryl moiety, or heteroaliphatic moiety;
—B1 is -T-L^{B1}-fucose,
wherein L^{B1} is a covalent bond or a group derived from the covalent conjugation of a T with an X;
—B2 is -T-L^{B2}-X
wherein X is a ligand comprising a saccharide, which may be fucose, mannose, or glucose; and L^{B2} is a covalent bond or a group derived from the covalent conjugation of a T with an X; and,
wherein n is 1, 2, or 3.

15. The pharmaceutical formulation of claim 1, wherein the insulin oligosaccharide conjugate comprises an insulin oligosaccharide conjugate selected from the group consisting of IOC-1, 1OC-2, IOC-3, IOC-4, IOC-5, IOC-6, IOC-7, IOC-8, IOC-9, IOC-10, IOC-11, IOC-12, IOC-13, IOC-14, IOC-15, IOC-16, IOC-17, IOC-18, IOC-19, IOC-20, IOC-21, IOC-22, IOC-23, IOC-24, IOC-25, IOC-26, IOC-27, IOC-28, IOC-29, IOC-30, IOC-31, IOC-32, IOC-33, IOC-34, IOC-35, IOC-36, IOC-37, IOC-38, IOC-39, IOC-41, IOC-42, IOC-43, IOC-44, IOC-45, IOC-46, IOC-47, IOC-49, IOC-50, IOC-51, IOC-52, IOC-53, IOC-54, IOC-55, IOC-56, IOC-57, IOC-58, IOC-59, IOC-60, IOC-61, IOC-62, IOC-63, IOC-64, IOC-65, IOC-66, IOC-67, IOC-68, IOC-69, IOC-70, IOC-71, IOC-72, IOC-73, IOC-74, IOC-75, IOC-76, IOC-77, IOC-78, IOC-79, IOC-80, IOC-81, IOC-82, IOC-83, IOC-84, IOC-85, IOC-86, IOC-87, IOC-88, IOC-89, IOC-90, IOC-91, IOC-92, IOC-93, IOC-94, IOC-95, IOC-96, IOC-97, IOC-98, IOC-99, IOC-100, IOC-101, 1OC-102, IOC-103, IOC-104, IOC-105, IOC-106, IOC-107, IOC-108, IOC-109, IOC-110, IOC-111, IOC-112, IOC-113, IOC-114, IOC-115, IOC-116, IOC-117, IOC-118, IOC-119, IOC-120, IOC-121, IOC-122, IOC-123, IOC-124, IOC-125, IOC-126, IOC-127, IOC-128, IOC-129, IOC-130, IOC-131, IOC-132, IOC-133, IOC-134, IOC-135, IOC-136, IOC-137, IOC-138, IOC-139, IOC-140, IOC-141, IOC-142, IOC-143, IOC-144, IOC-145, IOC-146, IOC-147, IOC-149, IOC-150, IOC-151, IOC-152, IOC-153, IOC-154, IOC-155, IOC-156, IOC-157, IOC-158, IOC-159, IOC-160, IOC-161, IOC-162, IOC-163, IOC-164, IOC-165, IOC-166, IOC-167, IOC-168, IOC-169, IOC-170, IOC-171, IOC-172, IOC-173, IOC-174, IOC-175, IOC-176, IOC-177, IOC-178, IOC-179, IOC-180, IOC-181, IOC-182, IOC-183, IOC-184, IOC-185, IOC-186, IOC-187, IOC-188, IOC-189, IOC-190, IOC-191, IOC-192, IOC-193, 1OC-194, IOC-195, IOC-196, IOC-197, IOC-198, IOC-199, IOC-200, IOC-201, IOC-202, IOC-203, IOC-204, IOC-205, IOC-206, IOC-207, IOC-208, IOC-210, IOC-211, IOC-212, IOC-213, IOC-214, IOC-215, IOC-216, IOC-217, IOC-218, IOC-219, IOC-220, IOC-221, IOC-222, IOC-223, IOC-224, IOC-225, IOC-226, IOC-227, IOC-228, IOC-229, IOC-230, IOC-231, IOC-232, IOC-233, IOC-234, IOC-235, IOC-236, IOC-237, IOC-238, IOC-239, IOC-240, IOC-241, IOC-242, IOC-243, IOC-244, IOC-245, IOC-246, IOC-247, IOC-248, IOC-249, IOC-250, IOC-251, IOC-252, IOC-253, IOC-254, IOC-255, IOC-256, IOC-257, IOC-258, IOC-259, IOC-260, IOC-261, IOC-262, IOC-263, IOC-264, IOC-265, IOC-266, IOC-267, IOC-268, IOC-269, IOC-270, IOC-271, and IOC-272.

16. The pharmaceutical formulation of claim 1, wherein the insulin oligosaccharide conjugate comprises an insulin oligosaccharide conjugate having the structure 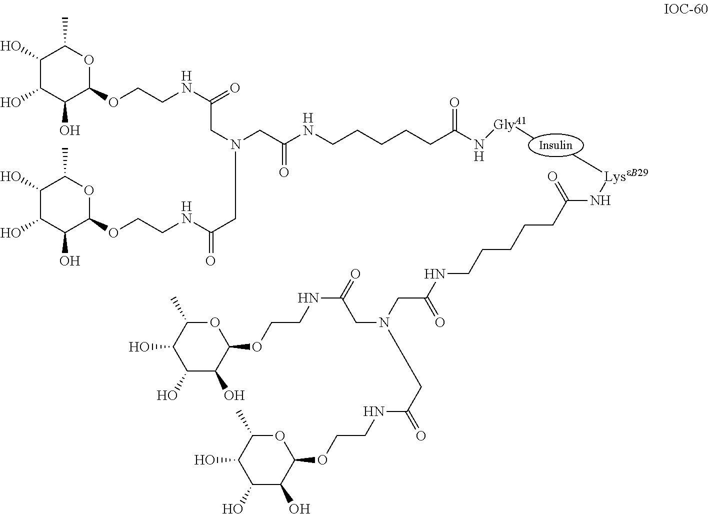
wherein the insulin is recombinant human insulin.

17. A method for controlling post-prandial glucose levels in an individual, comprising administering to the individual the formulation of claim 1 at a particular time before a meal to control the post-prandial glucose levels in the individual.

18. A method of treating an individual having diabetes comprising administering to the individual the formulation of claim 1 to treat the diabetes.

19. The method of claim 19, wherein the diabetes comprises Diabetes type I, Diabetes type II, or gestational diabetes.

20. A pharmaceutical formulation of claim 1 for controlling post-prandial glucose levels in the individual.

21. A pharmaceutical formulation of claim 1 for treatment of diabetes.

22. The pharmaceutical formulation of claim 16, wherein the diabetes comprises Diabetes type I, Diabetes type II, or gestational diabetes.

23-25. (canceled)


SEQUENCE LISTING (10 sequences)

SEQ ID NO: 1 - protein, 21 aa, Homo sapiens
GIVEQCCTSICSLYQLENYCN

SEQ ID NO: 2 - protein, 30 aa, Homo sapiens
FVNQHLCGSHLVEALYLVCGERGFFYTPKT

SEQ ID NO: 3 - protein, 30 aa, artificial sequence
  Insulin lispro B-chain
FVNQHLCGSHLVEALYLVCGERGFFYTKPT

SEQ ID NO: 4 - protein, 30 aa, artificial sequence
  Insulin aspart B-chain
FVNQHLCGSHLVEALYLVCGERGFFYTDKT

SEQ ID NO: 5 - protein, 30 aa, artificial sequence
  Insulin glulisine B-chain
FVKQHLCGSHLVEALYLVCGERGFFYTNKT

SEQ ID NO: 6 - protein, 21 aa, artificial sequence
  Insulin glargine A-chain
GIVEQCCTSICSLYQLENYCG

SEQ ID NO: 7 - protein, 32 aa, artificial sequence
  Insulin glargine B-chain
FVNQHLCGSHLVEALYLVCGERGFFYTPKTRR

SEQ ID NO: 8 - protein, 21 aa, artificial sequence
  Analog of the insulin A chain
  MISC_FEATURE (8)..(8): Xaa at position 8 is threonine or histidine
  MISC_FEATURE (21)..(21): Xaa at position 21 is asparagine, glycine, alanine, glutamine, glutamate, threonine, or serine
  MISC_FEATURE (21)..(21): Xaa is asparagine, glycine, alanine, glutamine, glutamate, threonine, or serine
GIVEQCCXSICSLYQLENYCX

SEQ ID NO: 9 - protein, 21 aa, artificial sequence
  Analog of the insulin B chain
  MISC_FEATURE (1)..(1): Xaa at position 1 is histidine or threonine
  MISC_FEATURE (5)..(5): Xaa at position 5 is alanine, glycine or serine
  MISC_FEATURE (6)..(6): Xaa at position 6 is histidine, aspartic acid, glutamic acid, homocysteic acid or cysteic acid
  MISC_FEATURE (6)..(6): Xaa is histidine, aspartic acid, glutamic acid, homocysteic acid or cysteic acid
XLCGXXLVEALYLVCGERGFF

SEQ ID NO: 10 - protein, 32 aa, artificial sequence
  Analog of the insulin B chain
  MISC_FEATURE (1)..(1): Xaa at position 1 is phenylalanine or desamino- phenylalanine
  MISC_FEATURE (5)..(5): Xaa at position 5 is histidine and threonine
  MISC_FEATURE (9)..(9): Xaa at position 9 is alanine, glycine or serine
  MISC_FEATURE (10)..(10): Xaa is histidine, aspartic acid, glutamic acid, homocysteic acid or cysteic acid
  MISC_FEATURE (28)..(29): Xaa for position 28 and Xaa for position 29 are selected from aspartate-lysine, lysine-proline, and a proline- lysine, respectively
  MISC_FEATURE (30)..(30): Xaa is threonine or alanine
  MISC_FEATURE (31)..(32): Xaa at positions 31-32 are either each arginine or each absent when position 30 is threonine
XVNQXLCGXXLVEALYLVCGERGFFYTXXXXX